SEQUENCE LISTING (7 sequences)

SEQ ID NO: 1 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 410 aa, Homo sapiens
MAEPRQEFEVMEDHAGTYGLGDRKDQGGYTMHQDQEGDTDAGLKESPPQTPTEDGSEEPGSETSDAKSTPTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 412 aa, Homo sapiens [sequence omitted]

SEQ ID NO: 6 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 42 aa, Homo sapiens [sequence omitted]

(19) United States
(12) Patent Application Publication
(10) Pub. No.: US 2008/0220449 A1
(43) Pub. Date: Sep. 11, 2008

(54) Biomarkers and assays for Alzheimer's disease

(75) Inventors: Sara Vasan, New York, NY (US); Eliot J. Davidowitz, West Hempstead, NY (US); James G. Moe, Stamford, CT (US)

Correspondence Address: KALOW & SPRINGUT LLP, 488 MADISON AVENUE, 19TH FLOOR, NEW YORK, NY, 10022, US

(73) Assignee: OLIGOMERIX, INC., New York, NY (US)

(21) Appl. No.: 12/069,399

(22) Filed: Feb. 8, 2008

Related U.S. Application Data
(60) Provisional application No. 60/900,205, filed on Feb. 8, 2007.

(51) Int. Cl. G01N 33/53; G01N 33/00; C07K 16/18; G01N 33/566

(52) U.S. Cl. 435/7.9; 436/86; 436/501; 530/388.1

(57) ABSTRACT

Methods, compositions and systems are provided for diagnosing, stratifying, or monitoring the progression or regression of Alzheimer's disease (AD), the methods, compositions and systems comprise detecting in a sample a level of at least one AD biomarker, the AD biomarker comprising at least phosphorylated tau pT217, soluble tau oligomer, tau-amyloid-beta 1-42 complex, a fragment thereof or a combination thereof and comparing the level from the sample to a reference level of phosphorylated tau pT217, soluble tau oligomer, and/or tau-amyloid-beta 1-42 complex to diagnose or stratify or monitor the progression or regression of AD. In various embodiments, diagnostic assay and screening kits are provided. In various embodiments, the assay and kits provided can monitor the therapeutic effect of a drug and/or AD treatment. In various embodiments, the assay can be used to screen for drugs that disrupt the AD biomarker(s)

This application claims the benefit of the filing date of Provisional Application No. 60/900,205, filed Feb. 8, 2007, entitled “Biomarkers for Alzheimer's Disease and Assays Therefor” this entire disclosure is hereby incorporated by reference into the present disclosure.

### BACKGROUND

Alzheimer's disease (AD) is the most common cause of dementia in the elderly that affects an estimated 15 million people worldwide and 40% of the population above 85 years. The disease is characterized by progressive loss of memory, speech and movement with a total incapacitation of the patient and eventually death. AD takes a terrible toll on those with the disease as well as their families, friends and caregivers.

The symptoms of AD manifest slowly and the first symptom may only be mild forgetfulness. In this stage, individuals may forget recent events, activities, the names of familiar people or things and may not be able to solve simple math problems. As the disease progresses into moderate stages of AD, symptoms are more easily noticed and become serious enough to cause people with AD or their family members to seek medical help. Moderate-stage symptoms of AD include forgetting how to do simple tasks such as grooming, and problems develop with speaking, understanding, reading, or writing. Severe stage AD patients may become anxious or aggressive, may wander away from home and ultimately need total care.

No cure is currently available for AD. Today, medication therapy focuses on controlling the symptoms of AD and its various stages. For example, mild to moderate AD can involve treatment with cholinesterase inhibitors such as Cognex® (tacrine), Aricept® (donepezil), Exelon® (rivastigmine), or Razadyne® (galantamine). Whereas moderate to severe AD can be treated with Namenda® (memantine). These medications may help delay or prevent AD symptoms from becoming worse for a limited period of time. So early AD treatment is warranted. However, there is no clear evidence that these medications have any effect on the underlying progression of the disease.

On the diagnostic side, there are currently no imaging tests (e.g., CT, MRI, MRA, etc.) to definitively diagnose AD while the patient is alive. Unfortunately, definitive diagnosis of AD is made by an autopsy of the brain tissue, which occurs after the patient's death.

When autopsied, studies of brain tissue have identified two major hallmarks of AD, the deposition of intracellular neurofibrillary tangles (NFT) and extracellular amyloid plaques (Binder L. I., et al., (2005) Biochim. Biophys. Acta. 1739:216-223; Josephs K A et al. Ann Neurol. In press (2007); Braak H, Braak E, Neurobiol Aging (1995) 16(3): 271-8). NFTs contain bundles of PHF (paired helical filaments), the major component of which is hyperphosphorylated tau, a microtubule associated protein. Amyloid plaque primarily contains the 42-amino acid form of β-amyloid protein (Aβ1-42) (Binder L. I., et al. (2005) Biochim. Biophys. Acta. 1739:216-223; Josephs K A et al., Ann Neurol. In press (2007); Braak H, Braak E, (1995) Neurobiol Aging. 16(3): 271-8).

Currently, there are no valid biomarkers identified in patient samples (e.g., cerebral spinal fluid, blood, urine, etc.) that can be used to specifically diagnose, stratify, or monitor the progression or regression of AD or other forms of dementia (e.g., Parkinson's disease, Huntington's disease, Creutzfeldt-Jakob disease (CJD), multiple-infarct dementia, etc.). The majority of AD biomarker studies are focused on the quantitative changes in tau and Aβ proteins and modifications of these proteins in the cerebral spinal fluid (CSF) from AD patients.

Levels of total tau (t-tau), phosphorylated tau (p-tau) and Aβ1-42 levels in the CSF have been extensively studied as potential biomarkers in AD. These studies have led to a consensus that an increase in total and p-tau and a concomitant decrease in Aβ1-42 in CSF may be indicative of AD. However, these changes in t-tau, p-tau, Aβ1-42 are not specific indicators of AD and also occur in some other forms of dementia (N. Andreasen et al., (2001) Arch Neurol. 58: 373-379; Formichi, P. et al., J. (2006) Cell. Physiol. 208: 39-46; Lewczuk P, et al., (2004) Neurobiol. Aging. 25:273-281; Sunderland T. et al., (2003) JAMA 289:2094-2103; Bailey P. Can. J. (2007) Neurol. Sci. 34: Suppl1 S72-S76; Blennow K., (2004) J. Am Soc. Exp. Neurotherapeutics. 1:213-225).

While extensive research in the past decade has identified possible biomarkers for AD, there is still an urgent need for biomarkers that are specifically useful in diagnosing, stratifying, or monitoring the progression or regression of AD. New biomarkers are also needed that serve as drug targets for the identification of new medication therapies to treat AD and to monitor different medications therapeutic effect when used to treat AD.

### SUMMARY

The present application provides methods and compositions for the identification of biomarkers associated with Alzheimer's disease (AD). Biomarkers identified according to the methods and compositions disclosed can be used in diagnostic and prognostic assays, allowing AD to be diagnosed earlier (while the patient is alive) and more accurately than was previously possible. The biomarkers can better help the clinician stratify, or monitor the progression or regression of AD, than currently available assays. In addition, biomarkers identified according to the composition and methods disclosed can serve as drug targets for the identification of new therapeutic agents for the treatment of AD and monitor different medication therapies benefit when used to treat AD.

In various embodiments, the present application provides AD biomarkers based on soluble tau oligomers that may act as early triggers of neurodegeneration, as well as, tau-Aβ complexes and phosphorylated tau pT217, and can aid in diagnosing, stratifying, or monitoring the progression or regression of AD. In various embodiments, AD biomarkers such as tau oligomers and tau-Aβ complexes are increased in severe AD, which allows, among other things, the differential diagnosis of severe AD from AD or non-AD. These biomarkers also provide novel tools to study the progression of the disease and monitor the effect of medication therapies.

In various embodiments, it has been discovered that increases in extracellular CSF AD biomarkers such as soluble tau oligomers, tau-Aβ complexes and/or phosphorylated tau pT217 occurs in AD and severe AD. Thus these AD biomarkers are useful tools which allows, among other things, the differential diagnosis of severe AD from AD or non-AD.

In one embodiment, a method of determining the presence or absence of Alzheimer's disease (AD) is provided, the method comprising detecting in a sample a level of an AD biomarker comprising at least one phosphorylated tau pT217, soluble tau oligomer, tau-Aβ1-42 complex or a fragment thereof or a combination thereof; and comparing the level from the sample to a reference level of phosphorylated tau pT217, tau oligomer, and/or tau-Aβ1-42 to determine the presence or absence of AD.

In another embodiment, a method of diagnosing, stratifying, or monitoring the progression or regression of Alzheimer's disease (AD) is provided, the method comprising detecting in a sample a level of at least one AD biomarker, the AD biomarker comprising at least phosphorylated tau pT217, soluble tau oligomer, tau-Aβ1-42 complex, a fragment thereof or a combination thereof and comparing the level from the sample to a reference level of phosphorylated tau pT217, tau oligomer, and/or tau-Aβ1-42 complex to diagnose or stratify or monitor the progression or regression of AD.

In yet another embodiment, a method of detecting or quantifying tau oligomer or tau-Aβ1-42 complex for use as a biomarker in Alzheimer's disease (AD) is provided, the method comprising contacting a sample with an antibody or fragment thereof and a labeled antibody or fragment thereof, the antibody or fragment thereof being capable of binding specifically to an epitope of tau or a fragment thereof and the labeled antibody or fragment thereof being capable of binding specifically to soluble tau oligomer, tau-Aβ1-42 complex or a fragment thereof or combination thereof; measuring a signal attributable to labeled antibody or fragment thereof bound to soluble tau oligomer or tau-Aβ1-42 complex; and correlating the measured signal to the presence or amount of soluble tau oligomer or tau-Aβ1-42 complex to use as a biomarker in AD.

In one exemplary embodiment, a method of detecting or quantifying soluble tau oligomer for use as a biomarker in Alzheimer's disease (AD) is provided, the method comprising contacting a sample with an antibody or fragment thereof and a labeled antibody or fragment thereof, the antibody or fragment thereof and the labeled antibody or fragment thereof being capable of binding specifically to the same epitope of soluble tau oligomer or a fragment thereof at different binding sites; measuring a signal attributable to bound labeled antibody or fragment thereof; and correlating the measured signal to the presence or amount of soluble tau oligomer to use as a biomarker in AD.

In a second exemplary embodiment, a diagnostic kit or assay useful for detecting tau oligomer, r tau-Aβ1-42 complex, and/or tau-Aβ 1-40 complex in Alzheimer's disease (AD) is provided, the kit or assay comprising: an antibody being capable of binding specifically to an epitope of soluble tau and a labeled antibody being capable of binding specifically to a conformational epitope of soluble tau oligomer or an epitope of tau-Aβ1-42 or an epitope of tau-Aβ1-40 complex.

In a third exemplary embodiment, a diagnostic kit or assay useful for detecting soluble tau oligomer in Alzheimer's disease (AD) is provided, the kit or assay comprising: a capture antibody and a labeled antibody, the capture antibody and the labeled antibody being the same antibody type and being capable of binding specifically to the same epitope of soluble tau oligomer at different sites.

In a fourth exemplary embodiment, a method of screening an agent for modulation or disruption of soluble tau oligomer is provided, the method comprising: a) contacting a sample containing soluble tau oligomer or a fragment thereof with an agent suspected of being capable of modulating tau oligomer formation or disrupting tau oligomers; and b) detecting the amount of soluble tau oligomer or fragment thereof in the sample, wherein a decrease in soluble tau oligomer indicates that the agent modulates tau oligomer formation or disrupts tau oligomer.

In a fifth exemplary embodiment, a method of monitoring a therapeutic effect of an agent that modulates or disrupts an AD (Alzheimer's disease) biomarker is provided comprising soluble tau oligomer, tau-Aβ1-42 complex, tau-Aβ1-40 complex, and/or fragment thereof, the method comprising: a) contacting a sample containing the AD biomarker with an agent suspected of being capable of modulating or disrupting the AD biomarker; and b) detecting the amount of the AD biomarker in the sample, wherein an increase or decrease in the AD biomarker indicates that the agent modulates soluble tau oligomer, tau-Aβ1-42 complex, tau-Aβ1-40 complex, and/or fragment thereof.

In a sixth exemplary embodiment, a method of monitoring progression of AD (Alzheimer's disease) in a patient is provided, the method comprising obtaining a sample of cerebral spinal fluid from the patient; detecting in the sample a level of an extracellular AD biomarker comprising soluble tau oligomer, tau-Aβ1-42 complex, tau-Aβ 1-40 complex, and/or fragment thereof, and determining if there is an increase or decrease in the level of AD biomarker thereby monitoring the progression of AD.

In a seventh exemplary embodiment, a system is provided comprising: a computing environment; an input device, connected to the computing environment, to receive data from a user, wherein the data received includes a level of an Alzheimer's disease (AD) biomarker comprising at least one phosphorylated tau pT217, soluble tau oligomer, tau-Aβ1-42 complex or a fragment thereof or a combination thereof from a cerebrospinal fluid sample; an output device, connected to the computing environment, to provide information to the user; and a computer readable storage medium having stored thereon at least one algorithm to provide for comparing the AD biomarker from the cerebrospinal fluid to an AD biomarker known to be indicative of the presence or absence of AD.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates schematic examples of formation of tau oligomer structures.

FIG. 2 illustrates the results obtained from an ELISA assay conducted to determine total tau levels in CSF from severe AD, AD and non-AD patients. Total tau protein was determined by sandwich ELISA formatted with monoclonal antibody (mAb) HT7 that specifically binds to amino acids 159-163 of tau 441 and found in all 6 tau isoforms in the central nervous system (CNS), and biotinylated mAb BT2 as reporter antibody that specifically binds to amino acids 194-198 of tau 441 and found in all 6 tau isoforms in the CNS.

FIGS. 3-5 illustrate the results obtained from an ELISA conducted to determine phosphorylated tau levels in CSF from severe AD, AD and non-AD patients. Phosphorylation of tau was determined by a sandwich ELISA using capture antibodies specific for tau phosphoepitopes at amino acid positions pS199/202; pT217; and pT231. The reporter antibody was biotinylated mAb HT7.

FIG. 6 is a schematic diagram illustrating standard and modified ELISA formats to detect tau monomers and oligomers.

FIG. 7 illustrates the results obtained from a modified ELISA conducted to determine tau-oligomer levels in CSF from severe AD, AD and non-AD patients. Tau oligomer levels were determined using a modified ELISA. The capture antibody was mAb HT7 that specifically binds to amino acid positions 159-163 of tau. The reporter antibody was biotinylated mAb HT7.

FIG. 8 illustrates the results obtained by ELISA using both tau and oligomer conformation-specific antibodies to determine tau oligomer levels in CSF from severe AD, AD and non-AD patients. The capture antibody was anti-tau mAb HT7, and the reporter antibody was polyclonal antibody (pAb) A11, specific for oligomer conformation.

FIG. 9 illustrates the results obtained by ELISA using both tau and oligomer conformation-specific antibodies to determine tau oligomer levels in CSF from severe AD, AD and non-AD patients. This ELISA was formatted with mAbT46 that specifically binds to the Carboxyl-terminus of the six tau isoforms in the CNS (c-terminal 37 amino acids) for capture, and pAb A11, specific for oligomer conformation, for reporter.

FIG. 10 illustrates the results obtained using sandwich ELISA to determine levels of tau modified with advanced glycation end product (AGE) in CSF from severe AD, AD and non-AD patients. The capture antibody was mAb HT7, and the reporter antibody was a pAb specific for AGE.

FIG. 11 illustrates the results obtained using sandwich ELISA to determine tau oligomer levels containing carboxyl-terminal truncated tau in CSF from severe AD, AD and non-AD patients. The capture antibody used was mAb tauC3, which specifically binds to truncated tau at Asp 421. The reporter antibody was pAb A11.

FIG. 12 illustrates the results obtained using sandwich ELISA to determine tau-Aβ1-42 complex levels in CSF from severe AD, AD and non-AD patients. The capture antibody was mAb HT7, and the reporter antibody was a biotinylated pAb against Aβ1-42.

FIG. 13 illustrates reduction of tau oligomer levels by curcumin in CSF from three different patients with severe AD. Modified ELISA was performed using mAb HT7 as capture antibody and biotinylated mAb HT7 as reporter.

FIG. 14 illustrates reduction of tau oligomer levels by curcumin with two different assay formats using the same sample of CSF from a patient having severe AD. One ELISA was formatted with mAb HT7 for capture and biotinylated mAb HT7 as reporter. The second ELISA was formatted with mAb T46 for capture and pAb A 11 for reporter so that only tau oligomers containing tau without c-terminal truncation would be detected.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “an antibody” includes one, two, three or more antibodies.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

The references, mentioned in the specification, are incorporated herein by reference for all that they disclose.

The headings below are not meant to limit the disclosure in any way; embodiments under any one heading may be used in conjunction with embodiments under any other heading.

### AD Definitions

The present disclosure includes methods and compositions for the identification of biomarkers associated with Alzheimer's disease (AD). Biomarkers identified according to the methods and compositions disclosed can be used in diagnosing, stratifying, or monitoring the progression or regression of AD. The biomarkers may be used as drug targets to develop new drugs and monitor different medication therapies to treat AD.

“Alzheimer's patient”, “AD patient”, and all refer to an individual who has been diagnosed with AD (for example, by MMSE score or post mortem by autopsy or has been given a probable diagnosis of Alzheimer's disease (AD). AD includes individuals with a probable diagnosis of mild AD, moderate AD, or severe AD. Non-AD patient refers to a “normal” individual or sample from a “normal” individual who has or would be assessed by a physician as not having AD or mild cognitive impairment (MCI). In various embodiments, a non-AD patient may have a Mini-Mental State Examination (MMSE) (referenced in Folstein et al., J. Psychiatr. Res 1975; 12:1289-198) score or would achieve a MMSE score in the range of 27 or above or assessed by another mental examination method. On average people with Alzheimer's disease who do not receive treatment lose 2 to 4 points each year on the MMSE. An “individual” is a mammal, more preferably a human. Mammals include, but are not limited to, humans, primates, farm animals, sport animals, rodents and pets.

An “individual with mild AD” or “mild AD” is an individual who has been diagnosed with AD (for example, post mortem by autopsy) or has been given a diagnosis of probable AD. In various embodiments, this individual has either been assessed with the Mini-Mental State Examination (MMSE) (referenced in Folstein et al., J. Psychiatr. Res 1975; 12:1289-198) and scored 20-26 or would achieve a score of 20-26 upon MMSE testing or assessed by another mental examination method.

An “individual with moderate AD” or “moderate AD” is an individual who has been diagnosed with AD (for example, post mortem by autopsy) or has been given a diagnosis of probable AD. In various embodiments, this individual has either been assessed with the MMSE and scored 10-19 or would achieve a score of 10-19 upon MMSE testing or assessed by another mental examination method.

An “individual with severe AD” or “severe AD” is an individual who has been diagnosed with AD (for example, post mortem by autopsy) or has been given a diagnosis of probable AD. In various embodiments, this individual has either been assessed with the MMSE and scored below 10 or would achieve a score of below 10 upon MMSE testing or assessed by another mental examination method.

As used herein, methods for “aiding diagnosis” refer to methods that assist in making a clinical determination regarding the presence, or nature, of the AD or MCI, and may or may not be conclusive with respect to the definitive diagnosis. Accordingly, for example, a method of aiding diagnosis of AD can comprise measuring the amount of one or more AD biomarkers in a biological sample from an individual.

The term “stratifying” refers to sorting individuals into different classes or strata based on the features of AD. For example, stratifying a population of individuals with Alzheimer's disease involves assigning the individuals on the basis of the severity of the disease (e.g., mild, moderate, severe, etc.).

As used herein, the term “treatment” refers to the alleviation, amelioration, and/or stabilization of symptoms, as well as delay in progression of symptoms of a particular disorder. For example, “treatment” of AD includes any one or more of: elimination of one or more symptoms of AD, reduction of one or more symptoms of AD, stabilization of the symptoms of AD (e.g., failure to progress to more advanced stages of AD), and delay in progression (e.g., worsening) of one or more symptoms of AD, and regression (e.g., reverting back to the earlier stage of AD).

As used herein, the term “predicting” refers to making a finding that an individual has a significantly enhanced probability of developing AD. The term “prognosis” includes the likely outcome or course of AD.

By “therapeutic effect” or “therapeutic activity” or “therapeutic action” it is meant a desired pharmacological activity of the agent against soluble tau oligomer, tau-Aβ1-42 complex, and/or tau-Aβ1-40 complex for the treatment of AD, mild AD, moderate AD, and/or severe AD. For example, a drug (e.g., NSAID, statin, etc.) can be given to a patient with AD, mild AD, moderate AD, or severe AD and the level of extracellular CSF soluble tau oligomer, tau-Aβ1-42 complex, and/or tau-Aβ1-40 complex can be measured to determine if the treatment has the desired therapeutic effect of lowering extracellular CSF levels of soluble tau oligomer, tau-A1-42 complex, and/or tau-Aβ1-40 complex. If there is lowering of the CSF levels, the drug is having the desired therapeutic effect and this may lead to an alleviation of symptoms of AD. If the CSF levels are not reduced, then the dose of the drug can be increased, discontinued, or another agent may be added till the desired therapeutic effect is achieved.

### AD Biomarkers

Three AD biomarkers extensively studied include total tau (t-tau), phosphorylated tau (p-tau) and Aβ1-42 levels in cerebrospinal fluid (CSF) (N. Andreasen et al., (2001) Arch Neurol. 58: 373-379; Formichi, P. et al., (2006) J. Cell. Physiol. 208: 39-46; Lewczuk P, et al., (2004) Neurobiol. Aging. 25:273-281; Sunderland T. et al., (2003) JAMA 289:2094-2103; Bailey P. Can. J. (2007) Neurol. Sci. 34 suppl S72-S76; Blennow K., (2004) J. Am Soc. Exp. Neurotherapeutics. 1:213-225). These studies have led to a consensus that changes involving an increase in total and p-tau and a concomitant decrease in Aβ1-42 in CSF may be indicative of AD. However, these changes are not specific indicators of AD and also occur in some other conditions of dementia (e.g., Parkinson's disease, Huntington's disease, Creutzfeldt-Jakob disease (CJD), multiple-infarct dementia, etc.). Numerous studies have shown that AD is associated with a significant increase in t-tau in CSF. However, the level of t-tau in CSF was only moderate in AD when compared to conditions of intense neurodegeneration such as CJD. A marked decrease in the concentration of Aβ1-42 has been reported in CSF from AD compared to controls. However, reduction in CSF Aβ1-42 also occurs in Lewy body dementia FTD, CJD and ALS (N. Andreasen, et al., (2001) Arch Neurol. 58: 373-379; Formichi, P., et al., (2006) J. Cell. Physiol. 208:39-46).

Hyperphosphorylation of tau is characteristic of AD and has been explored as a promising AD biomarker. At least 30 ser-pro and ser-thr phosphorylation sites have been identified in tau, outside the microtubule binding domains. While a hyperphosphorylation site specific for AD has not been identified, numerous studies have shown that phosphorylation at threonine 231 may be more indicative of AD than other forms of dementia. (Sjogren M, et al., (2003) Clin. Chim. Acta 332:1-10; Hampel H., et al., (2005) Arch. Neurol.: 62: 770-773; Buerger K, et al., (2006) Brain 129:3035-3041; de Leon M J et al., (2004) J Intern Med. September: 256(3): 205-23.

Other biomarkers for AD utilize the ratio of p-tau to Aβ1-42 as a sensitive marker for discriminating patients with AD from healthy controls as well as from subjects with other neurological disorders (Hampel H., et al. (2005) Arch. Neurol.: 62: 770-773; Buerger K, et al. (2006) Brain 129:3035-3041; de Leon M J, et al. (2004) J Intern Med. September; 256(3):205-23; Eweres M, et al. (2007) Neurology: 68:2205-2212). Sensitivity and specificity of the ratios were reported to be much higher than CSF concentrations of t-tau, p-tau, Aβ1-42, or t-tau and Aβ1-42 combined. A longitudinal study of CSF biomarkers indicated that changes in levels of tau and Aβ1-42, pTau181 over the baseline were much higher between groups than in the same groups during the progression of the disease (Bouwman F. H., et al. (2007) Neurology, 69:1006-1011). Thus, current CSF biomarkers may not be sensitive to follow the progression of the disease.

Miscellaneous biomarkers have been reported for AD including CSF levels of proteins of neuronal inflammation and oxidative stress (Bailey P (2007) Can. J. Neurol. Sci. 34:suppl 1 S72-S76; Stefani A, et al. (2006) J. Neurol. Sci. 251(1-2): 124-8; de Jong D, et al. (2007) Clin. Chem Lab Med. 45:1421-1434; Shaw LM (2007) Nat Rev Drug Discov. 6(4): 295-303). At present, these miscellaneous biomarkers have not led to any conclusive results. However, simultaneous measurement of biomarkers (multiplexed quantification) has been found to improve the quality control of assay performance (Lewczuk P et al. (2007) Neurobiol Aging. In Press).

The search for an AD biomarker also extends to recent proteomic studies on using CSF and serum (Finehout E J et al. (2006) Annals of Neurology 13 Dec.: 1-10; Ray S, et al. (2007) Nature Medicine 13:1359-1362). While these studies are encouraging, they are currently in the experimental stage and extensive validation studies are needed to assess the clinical significance.

While others have considered soluble tau oligomers and tau-Aβ complexes as unimportant in AD pathology, Applicants have found that they can be useful biomarkers in AD. In various embodiments, the present application provides AD biomarkers based on soluble tau oligomers that act as early triggers of neurodegeneration and can aid in diagnosing, stratifying, or monitoring the progression or regression of AD. In various embodiments, AD biomarkers such as tau oligomers and tau-Aβ complexes are increased in severe AD, which allows, among other things, the differential diagnosis of severe AD from AD or non-AD. These biomarkers also provide novel tools to study the progression of the disease and monitor the effect of medication therapies.

In various embodiments, unlike the prior art that has not focused on extracellular soluble tau oligomers, phosphorylated tau pT217, and/or tau-Aβ complexes in the CSF, Applicants have found that soluble tau oligomers and/or tau-Aβ complexes are useful biomarkers in AD. By “extracellular”, it is meant outside the cell, as opposed to “intracellular”, which is inside the cell.

A “biomarker” includes, nucleotide(s), protein(s), peptide(s), protein-protein complexes or aggregates, protein-peptide complexes or aggregates, peptide-peptide complexes or aggregates, immunogenic fragments, or metabolite(s) (e.g., glycated, truncated, phosphorylated peptide, protein, complex, aggregates) whose presence, absence, or, level of expression is a measure of the progression or regression of AD or of the likelihood of developing AD. A biomarker may comprise a single nucleotide(s), protein, peptide, protein-protein complex, protein-peptide complex, peptide-peptide complex or metabolite, or it may comprise a plurality of proteins, peptides, complexes, immunogenic fragments and/or metabolites whose presence, absence, or levels of expression collectively provide a measure of the progression or regression of AD or of the likelihood of developing AD.

In various embodiments, a composition (e.g. AD biomarker, antibody, or fragment thereof, etc.) that is “isolated” or “in substantially isolated form” refers to a composition that is in an environment different from that in which the composition naturally occurs. In various embodiments, a composition, which is in substantially isolated form may be substantially purified. The term “substantially purified”, in various embodiments, refers to a composition (e.g. AD biomarker, antibody, or fragment thereof, etc.) that is removed from its natural environment and is usually at least 60% free, 65%, free, 70% free, 75% free and most preferably 80% to 90% free, or 95% to 99% free from other components with which it is naturally associated.

### Tau

In various embodiments of the present application, we disclose novel AD biomarkers that are based on soluble tau oligomers in CSF that can be early triggers of neurodegeneration, as well as, tau-Aβ association and tau phosphorylation at T217. Accumulating evidence in recent years emphasizes the role of soluble oligomers of Aβ1-42 and tau as primary contributing factors for neuronal damage in AD (Kirkitadze. M.D., et al. (2002) J. Neurosc. Res. 69:567-577; Walsh D M, Selkoe D J. (2007) J. Neurochem. 101(5): 1172-84; Taniguchi T., et al. (2005) FEBS Letters, 579, 5704-5712; Santacruz K, et al. (2005) Science. 309(5733): 476-81; Yoshiyama Y, et al. (2007) Neuron 53(3): 337-51; Andorfer C, et al. (2005) J. Neurosci. 25(22):5446-54; Berger Z, et al. (2007) J. Neurosci. 27(14): 3650-62). Studies with animal models have shown that neuronal death can occur in the absence of NFTs and the presence of NFTs does not necessarily lead to dementia (Taniguchi T., et al. (2005) FEBS Letters, 579, 5704-5712; Santacruz K, et al. (2005) Science. 309(5733): 476-81; Yoshiyama Y, et al. (2007) Neuron 53(3): 337-51; Andorfer C, et al. (2005) J. Neurosci. 25(22):5446-54).

Accumulation of early-stage oligomeric aggregated tau species is associated with the development of functional deficits during the pathogenic progression of tauopathy, and accumulation of pre-fibril granular oligomers correlates with Braak staging in post-mortem analysis of AD brains (Berger Z, et al. (2007) J. Neurosci. 27(14): 3650-62; Maeda S, et al. (2006) Neuroscience Res. 54:197-201). The role of misfolded tau in AD has been shown in a number of studies using antibodies specific for tau conformational epitopes. Levels of conformationally altered tau in postmortem neocortical specimen increased with progressing dementia in AD (Haroutunian V, et al. (2007) Neurobiology of Aging, 28: 1-7). The involvement of soluble tau as the primary causative factor of neurotoxicity in AD is supported by these findings. Tau has also been found to associate with Aβ (beta amyloid plaque) in brain tissue, and this interaction is thought to facilitate the aggregation of these proteins (Guo J-P et al. (2006) PNAS 103:1953-1958).

Tau protein exists in 6 isoforms of 352-441 amino acid residues in the adult brain (Goedert et al. (1989) Neuron, 3, 519-526). The term “tau protein” refers to any protein of the tau protein family including, but not limited to, native tau protein monomer, precursor tau proteins, tau peptides, tau intermediates, metabolites, tau derivatives that can be antigenic, or antigenic fragments thereof. Fragments include less than entire tau protein provided the fragment is antigenic and will cause antibodies or antibody binding fragments to react with the tau fragment.

Tau proteins are characterized as one family among a larger number of protein families which co-purify with microtubules during repeated cycles of assembly and disassembly (Shelanski et al. (1973) Proc. Natl. Acad. Sci. USA, 70, 765-768), and known as microtubule-associated-proteins (MAPs). The tau family in addition is characterized by the presence of a characteristic N-terminal segment which is shared by all members of the family, sequences of approximately 50 amino acids inserted in the N-terminal segment, which are developmentally regulated in the brain, a characteristic tandem repeat region consisting of 3 or 4 tandem repeats of 31-32 amino acids, and a C-terminal tail. In various embodiments tau protein has the following amino acid sequence shown SEQ ID NOS. 1-6. Embodiments of tau protein may have an amino acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical or substantially identical to the sequence given in SEQ ID NOS: 1-6.

As applied to any of the disclosed tau protein, peptides, tau-Aβ complexes, the term “substantially identical” means that two peptide or protein sequences, when optimally aligned, such as by the programs GAP or BESTFIT using default gap weights, share at least 70 percent sequence identity, such as at least 90 percent sequence identity, or at least 95 percent sequence identity, or at least 99 percent sequence identity. Residue positions, which are not identical, in various embodiments, differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. In various embodiments, conservative amino acids substitution groups are: valine-leucine-isoleucine, phenylalanine-tyrosine, lysine-arginine, alanine-valine, glutamic-aspartic, and asparagine-glutamine.

The amino acid sequences of tau protein isoforms are also described in Ballatore C, Lee V M, Trojanowski J Q, Nat Rev Neurosci. 2007 September; 8(9):663-72. Review; Hasegawa M., Neuropathology. 2006 October; 26(5):484-90; and D'Souza I, and Schellenberg G D, Biochim Biophys Acta. 2005 Jan. 3; 1739(2-3):104-15. These references, mentioned in the specification, are incorporated herein by reference for all that they disclose.

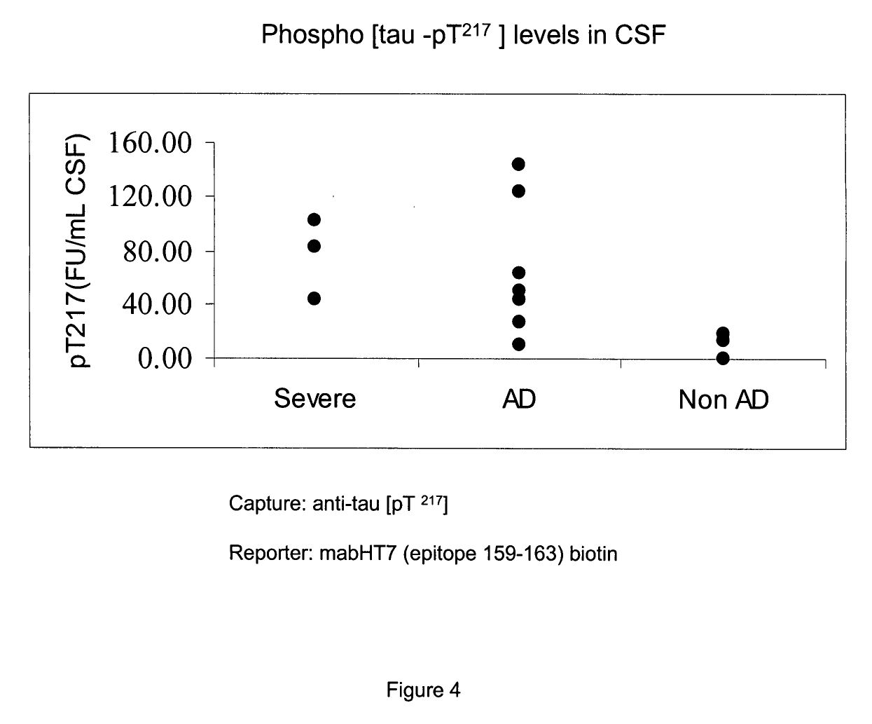

Tau proteins, peptides, or fragments thereof may undergo phosphorylation and there are over 21 phosphorylation sites. Tau is normally a phosphoprotein, but hyperphosphorylation of tau protein decreases its ability to promote microtubule assembly. Dephosphorylation of tau protein promotes rapid and extensive microtubule polymerization. Phosphorylated tau includes any of the tau proteins, its isoforms, precursor proteins, peptides, intermediates, derivatives, or immunologic fragments thereof that have been phosphorylated at one or more amino acid positions. One preferred phosphorylation site comprises phosphorylated tau pT217, which has been found to be a phosphorylation site that can be used as an AD biomarker or combined with other AD biomarkers in assays or methods for diagnosing, stratifying, or monitoring the progression or regression of AD. For example, one preferred assay is an ELISA assay that utilizes a capture antibody that specifically binds to the phosphorylation site of tau at amino acid position 217. This antibody can be combined with a reporter antibody, such as for example, labeled mAb HT7, which can bind to total tau at epitopes 159-163. This combination of reporter antibody and capture antibody in one assay can be utilized alone or combined with other AD biomarkers for diagnosing, stratifying, or monitoring the progression or regression of AD (FIG. 4).

In various embodiments, tau monomeric units (tau proteins (e.g., SEQ ID NOS: 1-6), peptides, or fragments thereof) may oligomerize and form tau oligomers, which are soluble in bodily fluids (e.g., CSF, blood, urine, cytoplasmic fluid, etc.). It has been found by Applicants that extracellular soluble tau monomeric units and/or tau oligomers increase in AD.

Tau oligomerization includes multimerizing two or more tau proteins, tau peptides, tau intermediates, tau metabolites, tau derivatives that can be antigenic, tau antigenic fragments, or tau-tau complexes. The multimer can contain any desired number of tau peptide/protein complexes and thus can form any multimer, such as but not limited to, a dimer, a trimer, a tetramer, a pentamer, a hexamer, octamer, decamer, dodecamer, or the like. However, in order to be soluble in bodily fluids (e.g., CSF, blood, urine, etc.) the multimer cannot be too long as it may become insoluble in bodily fluids. By “insoluble” is meant that the tau oligomer will precipitate out of the bodily fluid. In various embodiments, tau oligomer that comprises 50 tau monomer units is too long and may be insoluble in bodily fluids. In other embodiments, tau oligomer that comprises 100 tau monomer units is too long and may be insoluble in bodily fluids. By “soluble” is meant that the tau oligomer will dissolve in the bodily fluid. For example, soluble tau may be extracellular and appear in the CSF. By associated is meant covalent or non-covalent, hydrophobic or hydrophilic interactions, H bonding, or van der Waals attachment.

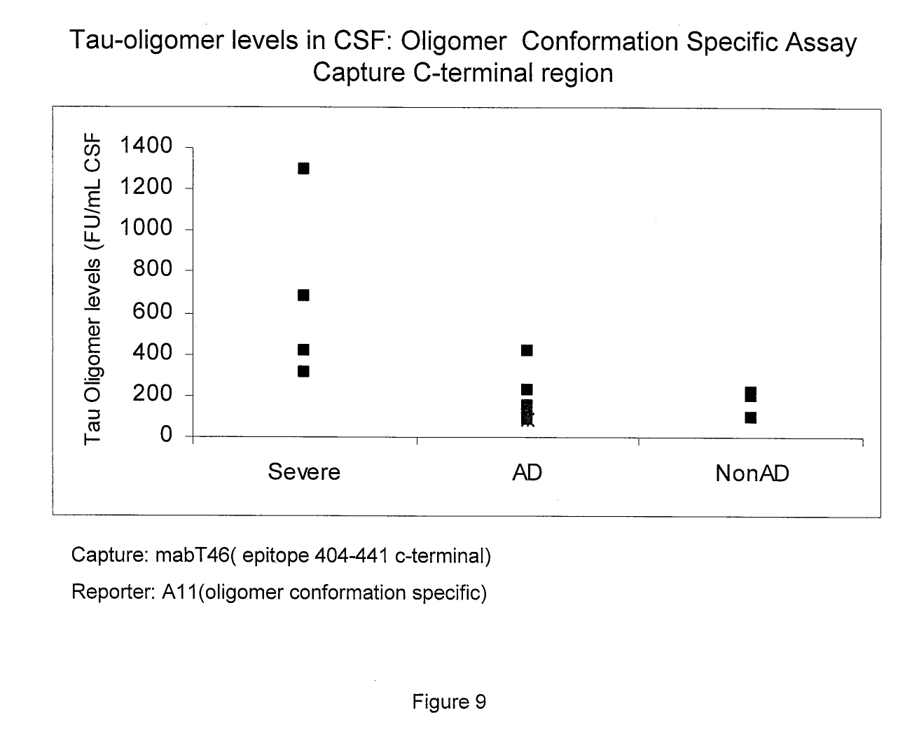

In various embodiments, soluble tau oligomer may be detected by one or more antibodies, such as for example, mAb HT7, A11, an oligomer conformation specific antibody, mabT46, or the like. In various embodiments, one preferred assay is an ELISA assay that utilizes a reporter antibody A11 that is specific for protein oligomer conformation. The capture antibody can be mAb T46 that specifically binds to C terminal amino acids 404-441 of tau 441 and found in all 6 tau isoforms expressed in the CNS. This combination of reporter antibody and capture antibody can be used alone to detect AD or combined with other AD biomarkers for diagnosing, stratifying, or monitoring the progression or regression of AD (FIG. 9).

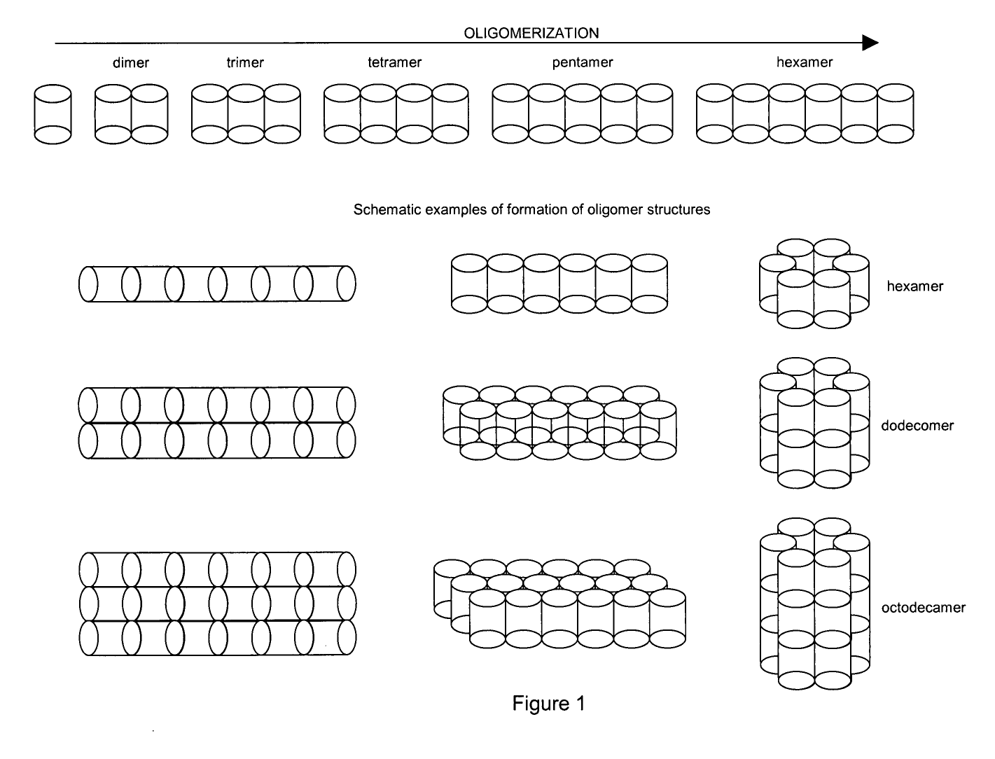

In vitro methods of making tau oligomer are described in U.S. application Ser. No. 11/704,079, filed Feb. 8, 2007 and U.S. Publication No. 20070218491, which is hereby incorporated by reference into the present disclosure. The method utilizes one or more polyanions to make tau oligomer and the application describes methods for quantifying tau oligomers by ELISA. FIG. 1 illustrates schematic examples of the formation of oligomer structures.

Specific examples of multimers, which may be utilized in accordance with certain embodiments are obtained from CSF or blood from patients having non-AD, AD, or severe AD. The definitive diagnosis in these samples was made post mortem by autopsy by examining brain tissue and determining NFTs and neuritic plaques.

Tau proteins, peptides, or fragments thereof may undergo glycation at one or more sites. Glycated tau includes any of the tau proteins, its isoforms, precursor proteins, peptides, intermediates, derivatives, or immunologic fragments thereof that have a carbohydrate molecule or multiple carbohydrate molecules (e.g., glucose) bound to one or more amino acid positions. Glycated tau proteins/peptides can be detected using an ELISA assay. For example, an ELISA assay was designed utilizing polyclonal antibody against AGE, which detects advanced glycation endproducts, in combination with a tau protein specific antibody. Polyclonal antibody against AGE can be combined with a capture antibody, such as for example, mAb HT7 that can bind an epitope of tau (e.g., amino acids positions 159-163). This combination of reporter antibody and capture antibody can be utilized alone or combined with other AD biomarkers for diagnosing, stratifying, or monitoring the progression or regression of AD (FIG. 9).

Tau proteins, peptides, or fragments thereof may undergo truncation at one or more sites (e.g., carboxy and/or amino truncations). For example, tau proteins may have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid truncation at the N and/or C terminus. Truncation typically occurs by enzymes, which cleave at the carboxy or amino terminus. Such enzymes, include, but are not limited to caspases that comprise cysteine proteases.

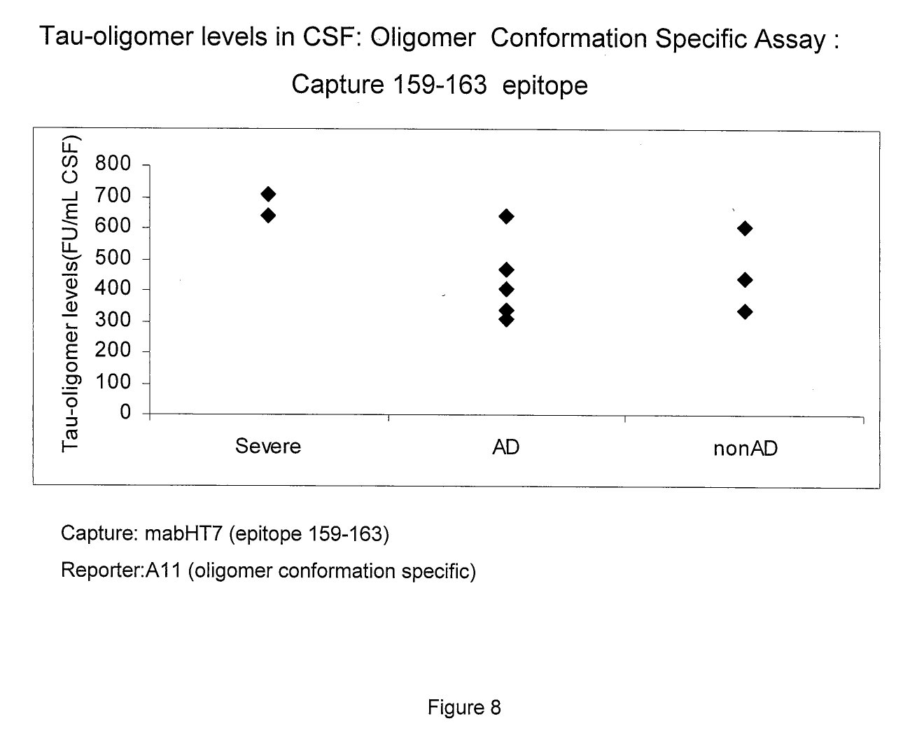

C-terminal ends of tau can be detected utilizing an ELISA assay. For example, one preferred capture antibody (mAb T46) to use in the ELISA assay binds to the C-terminal epitope of tau (e.g., 404-441). This capture antibody can be combined with A11 (polyclonal antibody specific for oligomer conformation) to be used as a reporter antibody. This combination of reporter antibody and capture antibody can be utilized alone or combined with other AD biomarkers for diagnosing, stratifying, or monitoring the progression or regression of AD (FIG. 8).

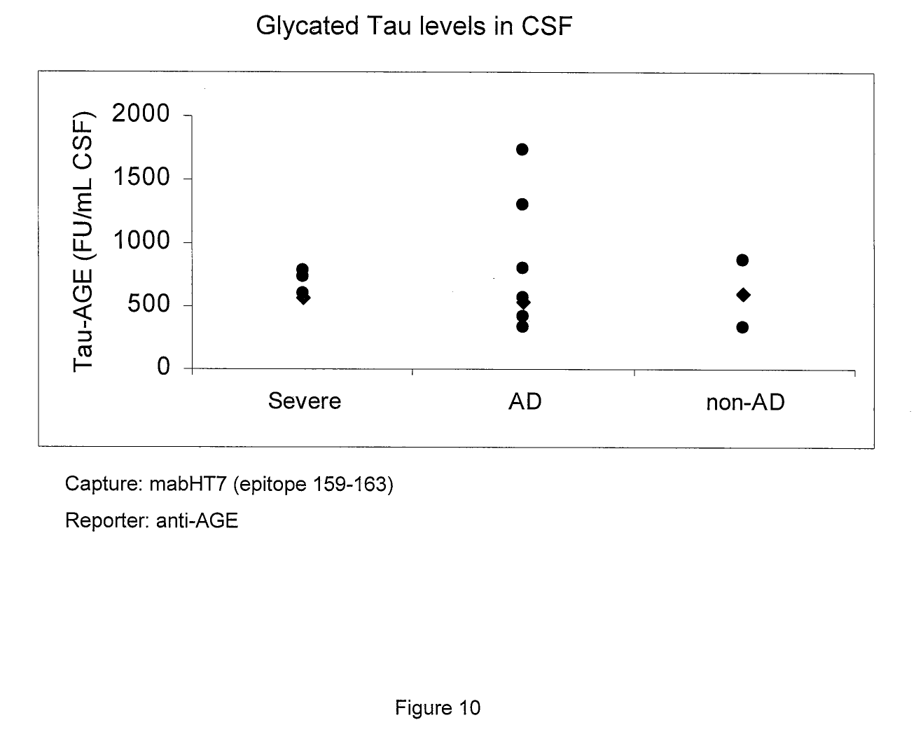

In another embodiment, the capture antibody employed is mAb tauC3, which specifically binds to truncated tau at position Asp 421. This capture antibody can be combined with A11 (polyclonal antibody specific for oligomer conformation), which is used as a reporter antibody. This combination of reporter antibody and capture antibody can be utilized alone or combined with other AD biomarkers for diagnosing, stratifying, or monitoring the progression or regression of AD (FIG. 10).

### Tau-Aβ1-42 Complex

In various embodiments, the methods and compositions disclosed utilize soluble tau-Aβ complex alone or combined with other AD biomarkers for diagnosing, stratifying, or monitoring the progression or regression of AD. By “soluble” is meant that the tau-Aβ complex will dissolve in the bodily fluid. The “soluble tau-Aβ complex” is extracellular and appears in bodily fluids (e.g., CSF, plasma, urine, etc.). Tau-Aβ complex includes interactions, aggregates, and/or coupling between tau, tau intermediates, metabolites, tau derivatives, or antigenic fragments of tau and β-amyloid protein (Aβ1-42), Aβ1-42 intermediates, Aβ1-42 metabolites, Aβ1-42 derivatives, or antigenic fragments of Aβ1-42. Aβ1-42 comprises 42 amino acids (SEQ ID NO: 7). In various embodiments, tau-AP complex comprises at least tau and β-amyloid protein (Aβ1-42) or tau and Aβ1-40 protein. In various embodiments, tau-Aβ complex comprises at least tau and P-amyloid protein (Aβ1-42) or tau and Aβ1-40 protein and tau and Aβ1-42 or Aβ1-40 molecules, such as, for example, amyloid oligomer, other amyloid protein, tau-oligomer-Aβ1-42 or tau and Aβ1-40 protein.

“Amyloid” refers to amyloidogenic proteins, peptides, or fragments thereof which can be soluble (e.g., monomeric or oligomeric) see, e.g., Lambert et al., Proc. Natl. Acad. Sci. U.S.A. 95, 6448-6453 (1998)). β-amyloid protein (Aβ) may comprise 39-43 amino acids. Typically, the Aβ1-42 peptide is produced by sequential proteolytic cleavage of the amyloid precursor protein (APP) by the enzyme(s) beta and gamma secretases. The length of the Aβ peptide appears to dramatically alter its biochemical/biophysical properties. Specifically, the additional two amino acids at the C-terminus of Aβ1-42 are very hydrophobic, presumably increasing the propensity of Aβ1-42 to aggregate. For example, Jarrett et al. demonstrated that Aβ1-42 aggregates vary rapidly in vitro as compared to Aβ1-40, suggesting that the longer forms of Aβ may be the important pathological proteins that are involved in the initial seeding of the neuritic plaques in Alzheimer's disease (Jarrett et al., Biochemistry 32, 4693-4697 (1993); Jarrett et al., Ann. NY Acad. Sci. 695, 144-148, (1993)).

As used herein, the term “β-amyloid” or “Aβ” or “amyloid beta” refer to amyloid beta proteins or peptides, amyloid beta precursor proteins or peptides, intermediates, and modifications and immunologic fragments thereof, unless otherwise specifically indicated. In particular, “Aβ” refers to any peptide produced by proteolytic processing of the APP gene product or peptides that are associated with amyloid pathologies, including Aβ1-39, Aβ1-40, Aβ1-41, Aβ1-42, and Aβ1-43. Embodiments of Aβ1-42 may have an amino acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical or substantially identical to the sequence given in SEQ ID NO: 7.

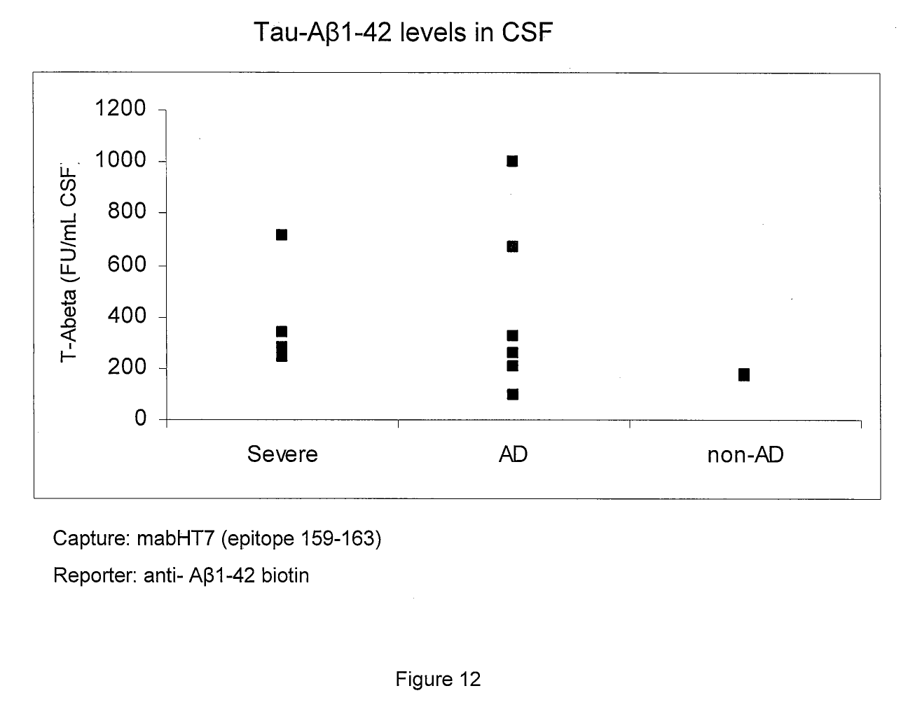

Tau-Aβ 1-42 complex can be detected utilizing methods known in the art. For example, one preferred method utilizes an ELISA assay to detect Aβ 1-42 complex. The reporter antibody utilized can be, for example, labeled anti-Aβ 1-42 specific for Aβ 1-42 and the capture antibody can be mAb HT7, which can bind to total tau at epitopes 159-163. This combination of reporter antibody and capture antibody can be utilized alone or combined with other AD biomarkers for diagnosing, stratifying, or monitoring the progression or regression of AD (FIG. 12). The Tau-Aβ 1-42 complex detected by this ELISA format may or may not contain molecules other than tau and Aβ 1-42.

### Detecting AD Biomarkers

In various embodiments, the method for detecting the presence or absence of AD in a subject includes detecting in a sample a level of an AD biomarker comprising at least one phosphorylated tau pT217, tau oligomer, tau-amyloid-beta 1-42 complex or a fragment thereof or a combination thereof, or any of the above in association with molecules other than tau and Aβ; and comparing the level from the sample to a reference level of phosphorylated tau pT217, tau oligomer, and/or tau-amyloid-beta 1-42 to determine the presence or absence of AD.

A sample may include, blood serum or blood plasma, CSF, urine and other liquid samples of biological origin from an individual or a set of individuals. Samples may have been manipulated in any way after their procurement, for example, by electrical, chemical and/or mechanical treatments. For example, the sample may be treated with reagents, solubilization, or enriched for certain components, such as proteins or peptides.

The AD biomarkers according to the methods and compositions provided can be detected by any suitable method. Detection paradigms that can be employed to this end include enzymatic methods, including immunological-based methods, optical methods, electrochemical methods (voltametry and amperometry techniques), atomic force microscopy, or radio frequency methods, e.g., multipolar resonance spectroscopy. It is to be understood that the present invention is not limited to a particular detection method. However, in some embodiments detection may be made by, for example, by fluorescent detection, spectrometric detection, chemiluminescent detection, matrix assisted laser desorption-time-of flight (MALDI-TOF) detection, high pressure liquid chromatographic detection, charge detection, mass detection, radio frequency detection, or light diffraction detection.

In various embodiments, detection of AD biomarkers can be accomplished using capture reagents specific to the AD biomarker (e.g., tau pT217, tau oligomer, tau-amyloid-beta 1-42 complex, etc.). In general, the capture reagent may be bound (e.g., covalently or non-covalently, via hydrophobic or hydrophilic interactions, H bonding, or van der Waals etc.) to a solid phase, such as a bead, a plate, a membrane or a chip. Methods of coupling biomolecules, such as antibodies or antigens, to a solid phase are well known in the art. They can employ, for example, bifunctional linking agents, or the solid phase can be derivatized with a reactive group, such as an epoxide or an imidizole, that will bind the molecule on contact. Capture reagents against different AD biomarkers can be mixed in the same place, or they can be attached to solid phases in different physical or addressable locations. For example, one can load multiple columns with derivatized beads, each column capable of capturing a single AD biomarker. Alternatively, one can pack a single column with different beads derivatized with capture reagents against a variety of AD biomarkers, thereby capturing all the analytes in a single place.

In various embodiments, biochips may be employed. The surfaces of biochips may be derivatized with the capture reagents directed against specific AD biomarkers. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound thereto. Thus, addressable arrays can be created to capture, detect and quantify one or more AD biomarkers.

### Immunoassays

In various embodiments, any of a variety of known immunoassay methods can be used for detection and quantification of the AD biomarker (e.g., tau pT217, tau oligomer, tau-amyloid-beta 1-42 complex, etc.), including, but not limited to, immunoassay, using an antibody specific for the encoded AD biomarker, e.g., by enzyme-linked immunosorbent assay (ELISA), radioimmunoassay (RIA), and the like; and functional assays for the encoded AD biomarker, e.g., binding activity or enzymatic activity.

Immunofluorescence assays can be easily performed on CSF. It is also possible to perform such assays in blood, serum or blood plasma, urine if sufficient AD biomarkers are diffused from human CSF to the plasma.

To increase the sensitivity of the assay, the immunocomplex (bound antibody and sample) may be further exposed to a second antibody (e.g., a reporter antibody), which is labeled and binds to the first antibody or to the biomarker. Typically, the secondary antibody comprises a detectably moiety, e.g., with a fluorescent marker so it can be easily visualized by any method (e.g., by eye, microscope, or machine).

As used herein, the terms “label” or “labeled” refers to incorporation of a detectable moiety on the protein, peptide, antibody, or fragment thereof, e.g., radiolabeled moiety, fluorescent label (e.g., FITC, rhodamine, lanthanide phosphors, dye quencher pairs, etc.), enzymatic label (e.g., horseradish peroxidase, beta-galactosidase, luciferase, alkaline phosphatase), chemiluminescent labels mass tags, histidine tags, chemiluminescent labels, or biotinyl moieties that can be detected by marked avidin (e.g., streptavidin or neutravidin containing a fluorescent marker or enzymatic activity that can be detected by optical or calorimetric methods). In some embodiments, labels are attached by spacer arms of various lengths to reduce potential steric hindrance with the support.

In one exemplary embodiment, to determine if the AD biomarker is present in CSF, samples from deceased patients, having AD, severe AD and control subjects having no AD was analyzed and showed similar trends with significant elevations in AD biomarkers comprising phosphorylated tau pT217, soluble tau oligomer, or tau-amyloid-beta 1-42 complex compared to controls (reference samples). These new biomarkers can be useful in diagnosing or stratifying or monitoring the progression or regression of AD. In this exemplary embodiment, the assay to detect and quantify these biomarkers utilized sandwich ELISA or modified ELISA.

Generally the sandwich ELISA or modified ELISA methods comprise contacting the sample with an antibody specific for an AD biomarker (e.g., tau pT217, soluble tau oligomer, or tau-amyloid-beta 1-42 complex, etc.). The antibody utilized may be any antibody, such as for example, monoclonal antibodies (e.g., mAb HT7, anti-tau[ps/ps¹⁹⁹⁻²⁰²], anti-tau [pT²¹⁷], anti-tau [pT²³¹], mAbT46, anti-truncAsp⁴²¹) immobilized to a support or it may be a polyclonal antibody that binds to more than one epitope. After allowing the sample time to bind with the antibody and washing of unbound sample, a labeled antibody is contacted with the sample or, in various embodiments, the capture antibody and sufficient time is allowed for the labeled antibody to specifically bind to the AD biomarker or the capture antibody. The bound label is detected and thus the AD biomarker is detected and can be quantified.

The level of antibody binding (either qualitative or quantitative) indicates the susceptibility of the patient to AD, mild AD, moderate AD, severe AD or non-AD. Examples of suitable reporter antibodies include labeled monoclonal antibodies (e.g., mAb HT7, anti-tau[ps/ps¹⁹⁹⁻²⁰²], anti-tau [pT²¹⁷], anti-tau [pT²³¹], mAb T46, anti-truncAsp⁴²¹) or fragments thereof and/or labeled polyclonal antibodies (e.g., A11, anti-AGE, anti-A beta 1-42) or fragments thereof.

For example, where the analyzed sample of AD biomarker is present at a level greater than that associated with a non-AD control, then the patient is susceptive to AD, mild AD, moderate AD, or severe AD. If the AD biomarker level is lower or equal to the non-AD control, then the patient is not susceptible to AD. In other embodiments, the control may be a severe AD control or a mild to moderate AD control, in which case if the analyzed AD biomarker level is lower than the control, then the patient is not susceptible to AD or has mild AD that has not progressed to severe AD. In other embodiments, if the control is an AD control (e.g., not severe AD, but AD), and if the AD biomarker level is higher than the control, then the patient is susceptible to severe AD or has severe AD. Medication therapies can be adjusted based on the AD biomarker level and disease progression or regression can be monitored. These embodiments illustrate ways that the assay can be used for diagnosing, stratifying, or monitoring the progression or regression of AD.

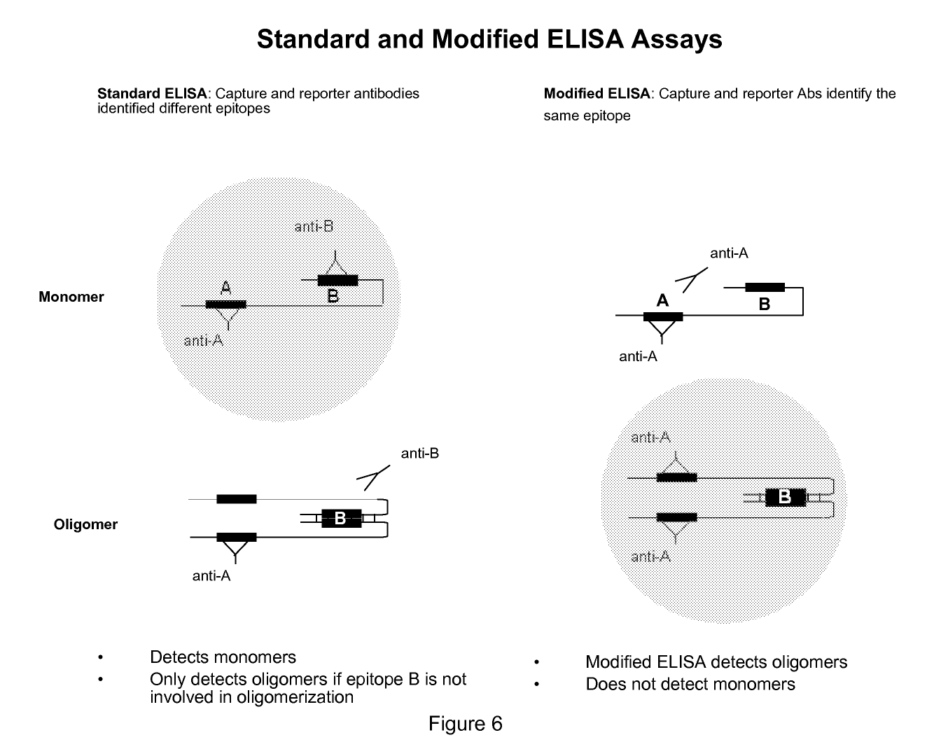

FIG. 6 is a schematic diagram illustrating an exemplary method and assay that utilizes standard and modified ELISA to detect tau-monomer, tau pT217, soluble tau oligomer, and tau-amyloid-beta 1-42 complex. Formation of tau oligomers was analyzed using two different ELISA methods to provide verified data. The “modified” ELISA uses the same monoclonal antibody for capture and detection. Insoluble aggregates are removed in the ELISA procedure during washing steps. Only soluble aggregates of tau, not monomers, are detected with this ELISA because the detection and reporter antibody recognizes an epitope which is present only once within tau. At least one more tau molecule must be present in the complex for detection with the same antibody. The modified ELISA, in various embodiments, used mAb HT7, which recognizes tau epitope amino acids 159-163, for capture and biotinylated mAb HT7 as the reporter antibody.

Unlike conventional assays, in various embodiments, the present assays and methods interrogate, among other things, soluble tau oligomer and/or tau-amyloid-beta 1-42 complexes. These molecules were generally considered uninformative in the prior art. However, Applicants have found that they can be useful biomarkers in AD.

In various embodiments, the methods and assays utilizes new platform formats for detecting these AD biomarkers. For example, in various embodiments, the assay and methods utilize an antibody that binds to one or more conformational epitopes of tau oligomer and/or tau-amyloid-beta 1-42 complexes. The assay also uses an antibody that binds to linear epitope. This allows for, in various embodiments, increased specificity and an assay that can supply information to diagnose AD, severe AD, or non-AD.

The term “epitope” or “antigenic determinant” refers to a site on an antigen to which B and/or T cells respond or a site on a molecule against which an antibody will be produced and/or to which an antibody will bind. For example, an epitope can be recognized by an antibody defining the epitope. A “linear epitope” is an epitope where an amino acid primary sequence comprises the epitope recognized. A linear epitope typically includes at least 3, and more usually, at least 5, for example, about 8 to about 10 amino acids in a unique sequence. A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the antibody defining the epitope). Typically a conformational epitope comprises an increased number of amino acids relative to a linear epitope. With regard to recognition of conformational epitopes, the antibody recognizes a 3-dimensional structure of the peptide or protein or fragment thereof. For example, when a protein molecule folds to form a three dimensional structure, certain amino acids and/or the polypeptide backbone forming the conformational epitope become juxtaposed enabling the antibody to recognize the epitope. Methods of determining conformation of epitopes include but are not limited to, for example, x-ray crystallography 2-dimensional nuclear magnetic resonance spectroscopy and site-directed spin labeling and electron paramagnetic resonance spectroscopy.

The term “antibody” is used to include intact antibodies and binding fragments thereof, including but not limited to, for example, full-length antibodies (e.g., an IgG antibody) or only an antigen binding portion (e.g., a Fab, F(ab′)₂ or scFv fragment). The fragment can also be generated by phage display technology known in the art.

Typically, fragments compete with the intact antibody from which they were derived for specific binding to an antigen. Optionally, antibodies or binding fragments thereof, can be chemically conjugated to, or expressed as, fusion proteins with other proteins. The term “antibody fragment” refers to a portion of a full-length antibody, generally the antigen binding or variable region. Examples of antibody fragments include Fab, Fab′, F(ab′)₂ and Fv fragments. In various embodiments, papain digestion of antibodies produces two identical antigen binding fragments, called the Fab fragment, each with a single antigen binding site, and a residual “Fc” fragment, so-called for its ability to crystallize readily. In various embodiments, pepsin treatment yields an F(ab′)₂ fragment that has two antigen binding fragments which are capable of cross-linking antigen, and a residual other fragment (which is termed pFc′). As used herein, “functional fragment” with respect to antibodies, refers to Fv, F(ab) and F(ab′)₂ fragments.

An “Fv” fragment is the minimum antibody fragment, which contains a complete antigen recognition and binding site. This region consists of a dimer of one heavy and one light chain variable domain in a tight, non-covalent association (V_{H}-V_{L} dimer). It is in this configuration that the three complementarity determining regions (CDRs) of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site. The Fab fragment (also designated as F(ab)) also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge-cysteines of the F(ab′)₂ pepsin digestion product. Additional chemical couplings of antibody fragments are known to those of ordinary skill in the art.

The term “monoclonal antibody” as used herein refers to an antibody obtained from a population of substantially homogeneous antibodies, e.g., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies, directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. “Polyclonal antibodies” include different antibodies directed against different determinants (epitopes).

“Specific binding” or “binding specifically” between two entities, may include a binding affinity of at least 10⁶, 10⁷, 10⁸, 10⁹ M⁻¹, or 10¹⁰ M⁻¹. Affinities greater than 10⁸ M⁻¹ are preferred for specific binding.

In various embodiments, the antibodies used in the methods and assays described and the binding sites for the antibodies that were used to analyze various CSF samples are listed in Table A.

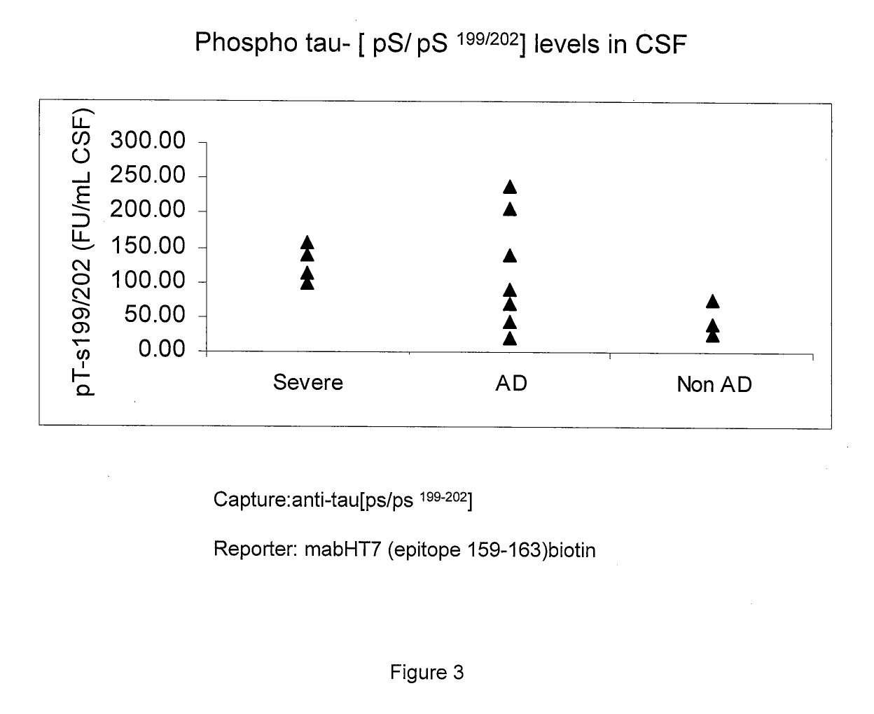

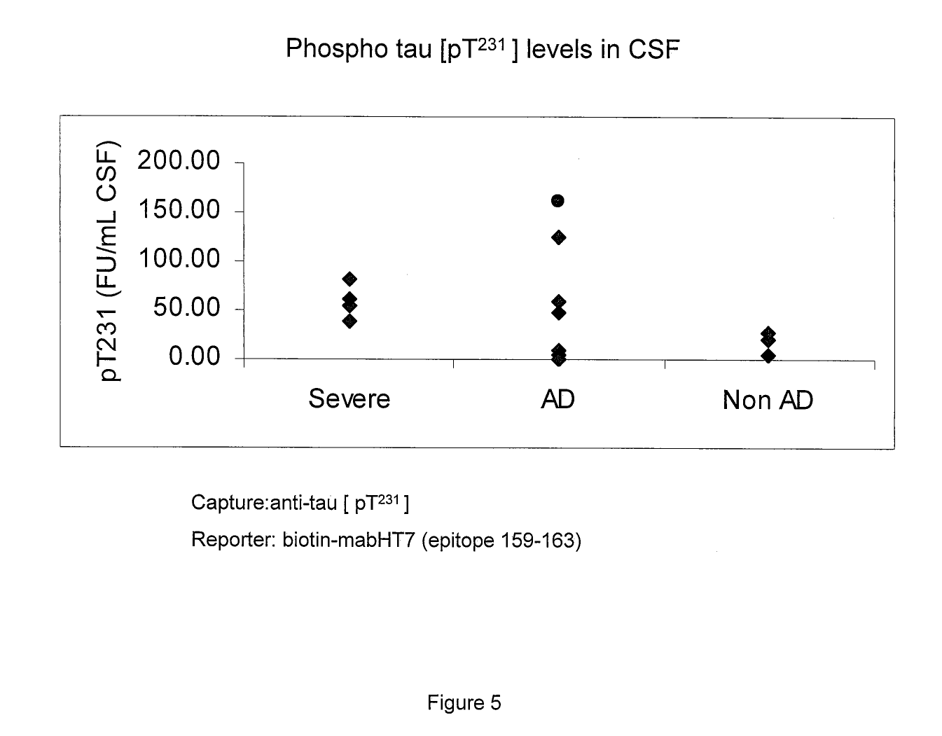

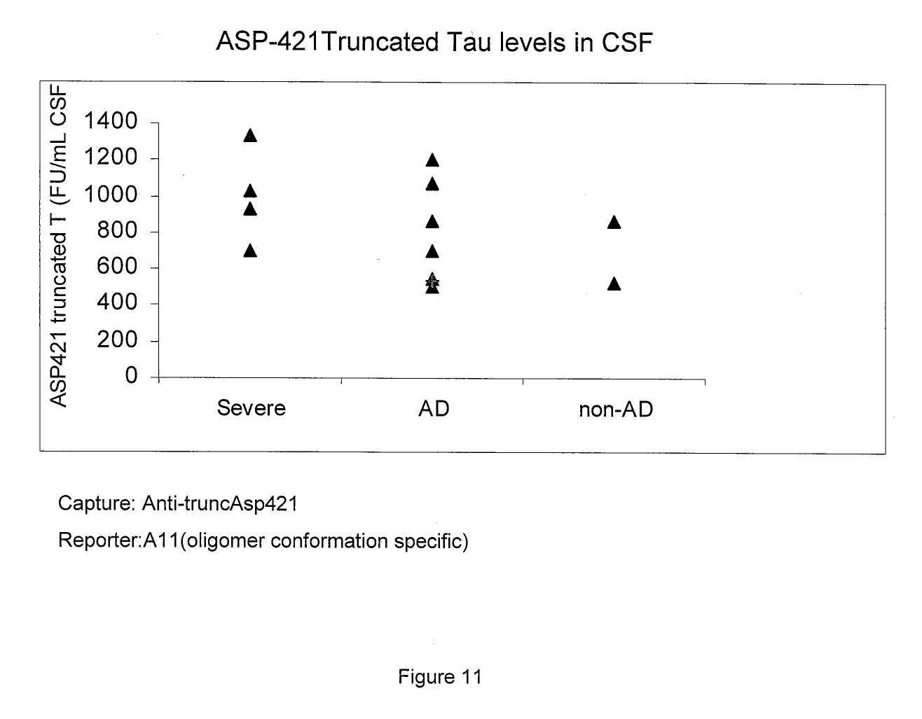

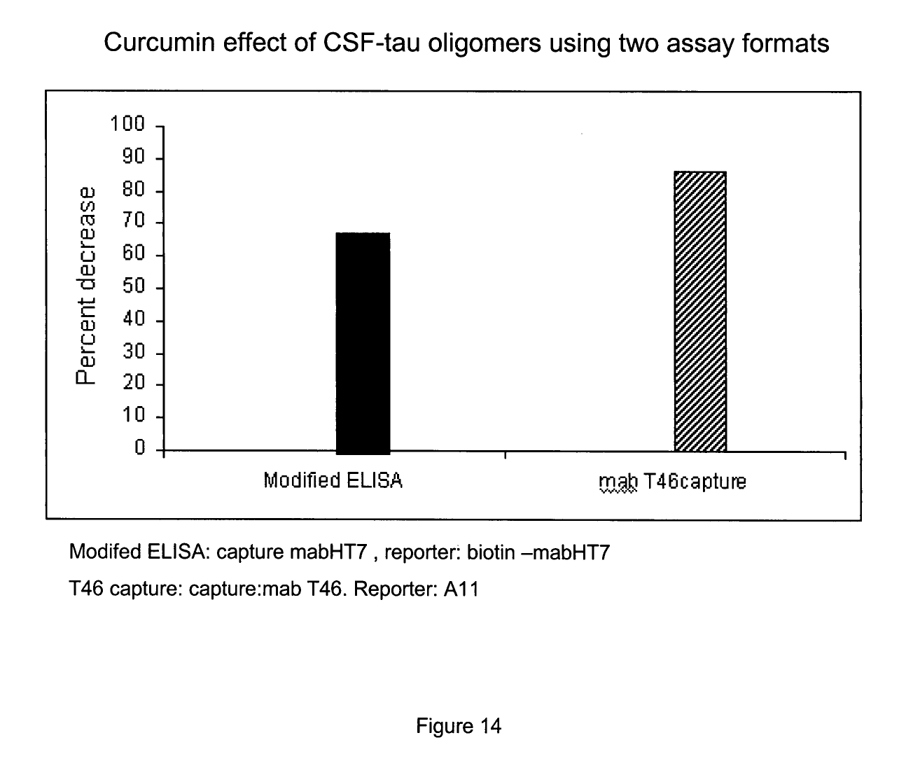

TABLE A

| Antibody | Epitope specificity | Application | Source |
|---|---|---|---|
| mAb HT7 | 159-163 | Capture antibody for total | Pierce (MN1000) |
| | (of Seq. ID No. 6) | tau, tau oligomer, glycated | |
| | | tau, (tau-Aβ1-42) assays. | |
| | | (FIGS.: 2, 7, 8, 10, 12-14.) | |
| Biotinylated mAb- | 159-163. | Reporter for modified | Pierce (MN1000B) |
| HT7 | (of Seq. ID No. 6) | ELISA (FIGS.: 7, 14) | |
| Biotinylated mAb | 194-198 | Reporter for standard | Pierce (MN1010 B) |
| BT2 | (of Seq. ID No. 6) | ELISA in total tau assay | |
| | | (FIG. 6) | |
| mAb T46 | 404-441 C-terminus | Capture antibody for tau- | Zymed/Invitrogen |
| | (of Seq. ID No. 6) | oligomer assay (FIGS. 9, 14) | ZY6400 |
| mAb TauC3 | Tau truncated at Asp | Capture antibody for | Chemicon (MAB5430) |
| | 421 | truncated tau oligomer | |
| | | assay.(FIG. 11) | |
| pAb | Tau phosphorylated at | Capture antibody for | Invitrogen (44-768G) |
| tau(pSpS¹⁹⁹/²⁰²) | Ser199 and Ser202 | tau(pSpS¹⁹⁹/²⁰²) (FIG. 3) | |
| pAb tau(pT²³¹) | Tau phosphorylated at | Capture antibody for | Invitrogen (44-746G) |
| | threonine 231 | tau(pT²³¹) assay (FIG. 5) | |
| pAb tau(pT²¹⁷) | Tau Phosphorylated | Capture antibody for | Invitrogen (44-744) |
| | at threonine 217 | tau(pT²¹⁷)assay (FIG. 4) | |
| Biotinylated pAb | Aβ1-42 (SEQ ID | Reporter for (tau-Aβ1-42) | Invitrogen (44-3449) |
| (anti-Aβ1-42) | NO. 7) | assay (FIG. 12, 13) | |
| pAb AGE | Glycated tau | Capture antibody for | Abcam(ab23722) |
| | | glycated tau assay (FIG. 10) | |
| pAb A11 | Oligomer | Reporter for tau oligomer | Invitrogen (AHB0052) |
| | conformation | assay (FIG. 8, 9, 11, 14) | |
| | specific antibody | | |
| Biotinylated anti- | | Detection antibody when | Pierce(31820) |
| rabbit | | non-biotinylated rabbit | |
| | | antibody was used as | |
| | | reporter. (FIGS.: 8-11, 14) | |

It will be understood by those of ordinary skill in the art that the binding sites or epitopes are based on SEQ ID NOs: 6 or 7 for convenience. These epitopes or binding sites may change as the reference sequence changes (e.g, SEQ ID NOS. 1-5, and 7). However, one of ordinary skill in the art can easily determine the binding site by, for example, aligning the reference sequence with SEQ ID NOs. 6 or 7 or sequences substantially identical to them. Moreover, the present application is not limited to one particular sequence, binding site(s) or epitope(s). Amino acid numbering will change based on insertions, deletions, transversions, and/or truncations, but the binding site or epitope can easily be determine by, for example, aligning the sequences and determining the epitope or binding site.

In one exemplary embodiment, the discriminatory power of the assay is illustrated in FIG. 4. An ELISA was conducted to determine phosphorylated tau levels in CSF from severe AD, AD and non-AD patients. Phosphorylation of tau was determined by a sandwich ELISA using capture antibodies specific for tau phosphorylated at phosphorylation site pT²¹⁷. The reporter antibody was biotinylated mAb HT7 specific for amino acids 159-163 of tau. Detection was done using HRP-neutravidin and QuantaBlu™ fluorogenic substrate, which allowed quantitation of greater than four optical density units. Patient samples were analyzed (3 with severe AD, 7 patients with AD and 3 patients having no AD as diagnosed by autopsy). The results in FIG. 4 from testing the sample with the assay clearly show that there is a large difference as measured by fluorescence units between patients with non-AD and severe AD. Thus tau pT²¹⁷ may be useful as a biomarker to diagnose AD from non-AD (e.g., determine the presence or absence of AD), or stratify or monitor the progression or regression of AD.

In another exemplary embodiment, the discriminatory power of the assay is illustrated in FIG. 9. A tau oligomer conformation ELISA (—COOH terminal capture) was conducted to determine tau oligomer levels in CSF from severe AD, AD and non-AD patients. The capture antibody used was mAb T46 that specifically binds to the 37 c-terminal amino acids of tau. The reporter antibody was pAb A 11, specific for tau oligomer conformation. Patient samples were analyzed (4 with severe AD, 4 patients with AD and 3 patients having no AD as diagnosed by autopsy). The results in FIG. 9 from testing the sample with the assay clearly show that there is a substantial difference, as measured by fluorescence units, between patients with non-AD and severe AD. Thus, this assay format can be used as a biomarker to diagnose AD from non-AD (e.g., determine the presence or absence of AD) or stratify or monitor the progression or regression of AD.

In yet another exemplary embodiment, the discriminatory power of the assay is illustrated in FIG. 12. A sandwich ELISA was performed to determine tau-Aβ1-42 complex levels in CSF from severe AD, AD and non-AD patients. The capture antibody was mAb HT7, which specifically binds amino acids 159-163 of tau. The reporter antibody was biotinylated pAb against Aβ1-42, which specifically binds Aβ1-42. The results in FIG. 12 from testing the sample with the assay clearly show that there is a substantial difference, as measured by fluorescence units, between patients with non-AD, AD and severe AD. Thus, this assay format can be used as a biomarker to diagnose AD from non-AD (e.g., determine the presence or absence of AD) or stratify or monitor the progression or regression of AD.

As will be readily apparent to the ordinarily skilled artisan upon reading the present application, the assay format and detection methods and other methods described herein can be varied. Such variations are within the intended scope of the invention. For example, in the above detection scheme, the AD biomarker can be immobilized on a solid support (e.g., an array), and then contacted with a labeled antibody to detect particular epitopes of the biomarker.

In various embodiments, a method of determining the presence or absence of AD is provided where the level of AD biomarker is detected by: (1) immobilizing the sample to a support and contacting the sample with one or more labeled antibodies or fragment thereof that specifically bind to an epitope of phosphorylated tau pT217, tau oligomer, tau-amyloid-beta 1-42 complex, or a fragment thereof or a combination thereof; (i) measuring a signal attributable to bound labeled antibody or fragment thereof; and (ii) correlating the measured signal to the presence or amount of phosphorylated tau pT217, soluble tau oligomer, and/or tau-amyloid-beta 1-42 complex; or (2) immobilizing the sample to a support and contacting the sample with one or more antibodies or fragment thereof that specifically bind to an epitope of phosphorylated tau pT217, soluble tau oligomer, tau-Aβ1-42 complex, or a fragment thereof or a combination thereof; (i) contacting the bound antibodies or fragment thereof with a labeled antibody or fragment thereof that specifically binds to the bound antibody or fragment thereof; (ii) measuring a signal attributable to bound labeled antibody or fragment thereof; and (iii) correlating the measured signal to the presence or amount of phosphorylated tau pT217, soluble tau oligomer, and/or tau-amyloid-beta 1-42 complex; or (3) immobilizing an antibody or fragment thereof to a support and contacting the sample with the immobilized antibody or fragment thereof so as to specifically bind an epitope of phosphorylated tau pT217, soluble tau oligomer, tau-amyloid-beta 1-42 complex, or a fragment thereof or a combination thereof; (i) contacting the bound sample with a labeled antibody or fragment thereof that specifically binds to a second epitope of phosphorylated tau pT217, soluble tau oligomer, and/or tau-amyloid-beta 1-42 complex; (ii) measuring a signal attributable to bound labeled antibody or fragment thereof; and (iii) correlating the measured signal to the presence or amount of phosphorylated tau pT217, tau oligomer (or tau protein in association with protein oligomer structure comprised of proteins other than tau), and/or tau-Aβ1-42 complex (composed of only tau and Aβ1-42 or in association with molecules other than tau or Aβ1-42).

The assays and methods can be provided as part of a kit. Thus, in various embodiments, diagnostic kits for detecting the presence/absence and/or a level of expression of an AD biomarker in a sample are provided. The kits may contain one or more antibodies (e.g., monoclonal antibodies, polyclonal antibodies, labeled and unlabeled), fragments thereof for detecting an AD biomarker. The kit may optionally provide additional components that are useful in the procedure, including, but not limited to, buffers, developing reagents, labels, reacting surfaces, means for detection, control samples, standards, instructions, and interpretive information.

### Mass Spectrometry

In various embodiments, the AD biomarker may be detected by mass spectrometry, or methods that employs a mass spectrometer to detect gas phase ions. Examples of mass spectrometers are time-of-flight, magnetic sector, quadrupole filter, ion trap, ion cyclotron resonance, electrostatic sector analyzer and hybrids of these. In such embodiments, the relative levels of AD biomarker in each sample can be determined with mass spectrometry where a standard curve can be generated using corresponding synthetic peptides without isotope labeling. Alternatively, the AD biomarker in the sample can be identified and quantified when the identical synthetic peptides are isotope labeled and spiked in the sample.

In certain embodiments, the mass spectrometer may be a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

In general, a probe with an adsorbent surface is contacted with the sample obtained from an individual for a period of time sufficient to allow AD biomarker that may be present in the sample to bind to the adsorbent surface. After an incubation period, the substrate is washed to remove unbound material. Any suitable washing solutions can be used; such as an aqueous solution. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. An energy absorbing molecule is then applied to the substrate with the AD biomarker. The AD biomarkers bound to the substrate are then detected in a gas phase ion spectrometer such as a time-of-flight mass spectrometer or an ion trap mass spectrometer. The AD biomarker is ionized by an ionization source such as a laser, the generated ions are collected by an ion optic assembly, and then a mass analyzer disperses and analyzes the passing ions. The detector then translates information of the detected ions into mass-to-charge ratios. Detection of an AD biomarker may involve the detection of the signal intensity. Thus, both the quantity and mass of the AD biomarker can be determined.

In another mass spectrometry method, the AD biomarker may be first captured on a chromatographic resin that binds it. For example, the resin can be derivatized with an antibody. Alternatively, this method could be preceded by chromatographic fractionation before application to the bio-affinity resin. After elution from the resin, the sample can be analyzed by MALDI, electrospray, or another ionization method for mass spectrometry. In another alternative, one could fractionate on an anion exchange resin and detect by MALDI or electrospray mass spectrometry directly. In yet another method, one could capture the AD biomarker on an immuno-chromatographic resin that comprises antibodies that bind the AD biomarker, wash the resin to remove unbound material, elute the bound molecules from the resin and detect the eluted proteins by MALDI, electrospray mass spectrometry or another ionization mass spectrometry method.

### Reference Levels and Comparisons

The reference level used for comparison with the measured level for an AD biomarker may vary, depending on the embodiment employed. For AD diagnosis methods, the “reference level” may be a predetermined reference level, such as an average of levels obtained from a population that is not afflicted with AD, but in some instances, the reference level can be a mean or median level from a group of individuals including AD patients. In some instances, the predetermined reference level is derived from (e.g., is the mean or median of) levels obtained from an age-matched population.

For AD monitoring methods (e.g., methods of diagnosing or aiding in the diagnosis of AD progression in an AD patient), the reference level may be a predetermined level, such as an average of levels obtained from a population that is not afflicted with AD or a population that has been diagnosed AD, and, in some instances, the reference level can be a mean or median level from a group of individuals including non-AD and/or AD patients. In some instances, the reference level may be derived from (e.g., is the mean or median of) levels obtained from an age-matched population.

For AD stratification methods (i.e., methods of stratifying AD patients into mild, moderate and severe stages of AD), the reference level may be normally a predetermined reference level that is the mean or median of levels from a population which has been diagnosed with AD or non-AD (preferably a population diagnosed with AD). In some instances, the predetermined reference level is derived from (e.g., is the mean or median of) levels obtained from an age-matched population.

The process of comparing a measured value and a reference value can be carried out in any convenient manner appropriate to the type of measured value and reference value for the AD biomarker at issue. The “measuring” of AD biomarker can be performed using quantitative or qualitative measurement techniques, and the mode of comparing a measured value and a reference value can vary depending on the measurement technology employed. For example, when a qualitative calorimetric assay is used to measure AD biomarker levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). However, it is expected that the measured values used in the methods will most commonly be quantitative values (e.g., quantitative measurements of concentration, such as nanograms of AD biomarker per milliliter of sample, or absolute amount). In other examples, measured values are qualitative. As with qualitative measurements, the comparison can be made by inspecting the numerical data, by inspecting representations of the data (e.g., inspecting graphical representations such as bar or line graphs). A measured value is generally considered to be substantially equal to or greater than a reference value if it is at least 95% of the value of the reference value (e.g., a measured value of 1.71 would be considered substantially equal to a reference value of 1.80). A measured value is considered less than a reference value (e.g., the control) if the measured value is less than 95% of the reference value (e.g., a measured value of 1.7 would be considered less than a reference value of 1.80). A measured value is considered more than a reference value if the measured value is at least more than 5% greater than the reference value (e.g., a measured value of 1.89 would be considered more than a reference value of 1.80).

The process of comparing analyzed sample with the reference value or control may be manual (such as visual inspection by the practitioner of the method) or it may be automated. For example, an assay device (such as a luminometer for measuring chemiluminescent signals) may include circuitry and software enabling it to compare a measured value with a reference value for an AD biomarker. Alternately, a separate device (e.g., a digital computer) may be used to compare the measured value(s) and the reference value(s). Automated devices for comparison may include stored reference values for the AD biomarker(s) being measured, or they may compare the measured value(s) with reference values that are derived from contemporaneously measured reference samples.

In some embodiments, the methods may utilize “simple” or “binary” comparison between the measured level(s) and the reference level(s) (e.g., the comparison between a measured level and a reference level determines whether the measured level is higher or lower than the reference level to give a plus or minus result). For AD diagnosis biomarkers, a comparison showing that the measured value for the biomarker is lower than the reference value indicates or suggests a diagnosis of AD.

In various embodiments, an increase in extracellular soluble tau oligomer, soluble phosphorylated tau pT217, soluble tau-Aβ1-42 complex, tau-Aβ 1-40 complex or a fragment thereof includes increases in levels from 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 855%-90%, 90%-95%, 95%-100%, or by 100%, 150%, 200%, 250%, 300%, 350%, 400%, 450%, 500%, 550%, 600%, 650%, 700%, 750%, 800%, 850%, 900%, 950%, 1000%, or higher when compared to the control. This increase (e.g., 5% increase in pg/ml of tau oligomer in the CSF over the control) may be seen in AD, and in severe AD or as the disease progresses or as treatment starts to fail.

In various embodiments, a decrease in extracellular soluble tau oligomer, soluble phosphorylated tau pT217, soluble tau-Aβ1-42 complex, tau-Aβ 1-40 complex or a fragment thereof includes decreases in levels from 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 855%-90%, 90%-95%, 95%-100%, or by 100%, 150%, 200%, 250%, 300%, 350%, 400%, 450%, 500%, 550%, 600%, 650%, 700%, 750%, 800%, 850%, 900%, 950%, 1000%, or lower when compared to the control. This decrease (e.g., 5% decrease in pg/ml of tau oligomer in the CSF over the control) may be seen as there is an improvement in the disease (e.g., from severe AD to moderate AD) or treatment begins to work.

In addition to diagnostic methods, the invention also features methods for monitoring the progression of AD in a patient. Such methods include obtaining a sample from the patient, detecting a level of extracellular soluble tau oligomer, tau-Aβ1-42 complex, tau-Aβ1-40 complex and/or fragment thereof. A second measurement of the level of extracellular soluble tau oligomer, tau-Aβ1-42 complex, tau-Aβ1-40 complex and/or fragment thereof is subsequently performed using the same steps following a time interval (e.g., at least 1, 2, 5, 7, 14, or 28 days, or at least 1, 2, 3, 4, 5, 6, 8, 10, 12, or 24 months). The two measurements are then compared, where an increase in levels is indicative of AD disease progression, while a decrease in levels is indicative of improvement.

Such monitoring methods can be performed in conjunction with administration of a therapy (e.g., pharmaceutical therapy) to the patient and, thus, can be used to determine if a particular therapy is having the desired effect on the AD biomarker, which can be indicative of the severity of the AD. In one embodiment, the first measurement is taken prior to commencement of a therapy. Therapy is begun following the first measurement, and a second measurement is performed (e.g., three to six months later) following the commencement of therapy. A change in the second measurement as compared to the first measurement can thus be taken as indication of the effectiveness of the therapy.

### Screening Compounds

In various embodiments, methods of screening for candidate agents for the treatment of AD are provided by assaying prospective candidate agents for activity in modulating AD biomarkers particularly tau oligomer.

The screening methods may utilize the AD biomarkers described herein as “drug targets.” Prospective agents are tested for activity in modulating a drug target in an assay system. As will be understood by those of skill in the art, the mode of testing for modulation activity will depend on the AD biomarker and the form of the drug target used (e.g., complete protein or peptide fragment).

When the AD biomarker protein/peptide itself is the drug target, prospective agents are tested for activity in modulating levels or activity of the protein/peptide itself (e.g., soluble tau oligomer). Modulation of levels of an AD biomarker can be accomplished by, for example, increasing or reducing half-life of the biomarker protein or drug candidate concentration. Modulation of activity of an AD biomarker can also be accomplished by increasing or reducing the availability of the AD biomarker to bind to its receptor(s) or ligand(s).

Screening assays may be in any format. Preferred formats include ELISA and modified ELISA formats shown in FIG. 6. In various embodiments, a method of screening an agent for modulation or disruption of soluble tau oligomer is provided, the method comprising: a) contacting a sample containing soluble tau oligomer or a fragment thereof with an agent suspected of being capable of modulating tau oligomer formation or disrupting tau oligomers; and b) detecting the amount of soluble tau oligomer or fragment thereof in the sample, wherein a decrease in soluble tau oligomer indicates that the agent modulates tau oligomer formation or disrupts tau oligomer. In various embodiments, the mechanism that the agent disrupts tau oligomer formation is by inhibiting tau-tau binding.

The screening assay can also be used to determine effective doses of agents to optimize disruption or modulation of tau oligomer. For example, the method of screening compounds may involve comparing the sample being analyzed to a sample that does not contain the agent or less agent to determine if the agent modulates or inhibits tau oligomer. “Disruption” of tau oligomer includes the interruption of tau oligomer formation.

Prospective agents for use in the screening methods may be chemical compounds and/or complexes of any sort, including both organic and inorganic molecules (and complexes thereof). As will be understood in the art, organic molecules are most commonly screened for AD biomarker modulatory activity. In some situations, the prospective agents for testing will include the target AD biomarker (e.g., soluble tau oligomer).

Suitable agents for screening include, but are not limited to, antioxidants (vitamin E and vitamin C), anti-inflammatory agents (e.g., curcumin, demethoxycurcumin, bis-demethoxycurcumin, and/or morin), antibiotics, chelation agents, clioquinol, ergoloids, estrogen, herbal agents (e.g., ginko biloba, huperzine A, melissa officinalis (lemon balm), etc.), statins, vitamin B, or combinations thereof or derivatives thereof. Derivatives include analogs of the above agents that are pharmacologically active.

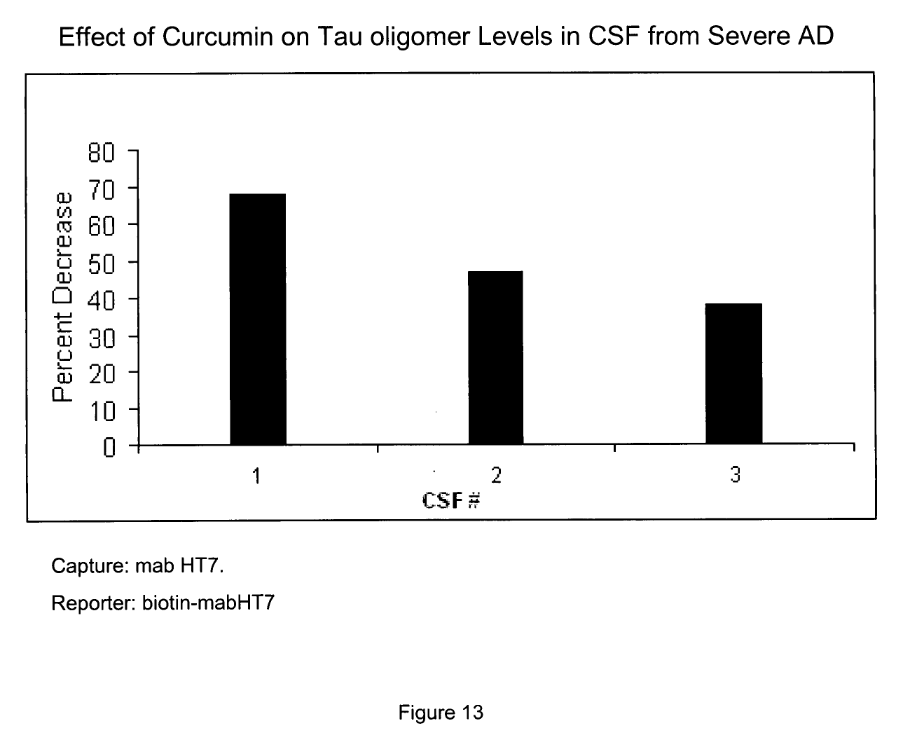

Curcumin is a known anti-inflammatory agent that exerts its effect on NF-KB and COX-2 while having little or no effect on COX-1. Curcumin can be synthesized by methods known in the art. FIG. 13 illustrates curcumin inhibition of tau oligomer in the CSF in three different patients having severe AD. Modified ELISA was performed using mAb HT7 as the capture antibody that specifically binds to amino acid positions 159-163 of tau. The reporter antibody was biotinylated mAb HT7.

FIG. 14 illustrates curcumin inhibition of tau oligomer in the CSF for two different assay formats in one sample of CSF from a patient having severe AD. One assay format utilized a modified ELISA using mAb HT7 as the capture antibody. The reporter antibody was biotinylated mAb HT7. The second assay format utilizes mAbT46, specific for the 37 c-terminal amino acids of tau, for capture, and pAb A11, specific for oligomer conformation, as reporter. Both formats measure curcumin's inhibition or disruption of tau oligomer.

### Computer Based Systems

In various embodiments, the methods and assays provided are suitable for automated systems for performing the methods described above using computer-readable instructions, e.g., programming, logic etc. Accordingly, in some embodiments, computer programming for analyzing and comparing a pattern of AD biomarkers in a sample obtained from a subject to a library of known AD biomarkers known to be somewhat indicative of the presence or absence of AD, wherein the comparing indicates the presence or absence of AD, non-AD or severe AD.

The methods and systems described herein can be implemented in numerous ways. In one embodiment of particular interest, the methods involve use of a communications infrastructure, for example the Internet. In various embodiments, the methods and systems provided may be implemented in various forms of hardware, software, firmware, processors, or a combination thereof.

In various embodiments, the methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility). For example, during or after data input by the user, portions of the data processing can be performed in the user-side computing environment. In various embodiments, the user-side computing environment can be programmed to provide for defined test codes to denote platform, carrier/diagnostic test, or both; processing of data using defined flags, and/or generation of flag configurations, where the responses are transmitted as processed or partially processed responses to the reviewer's computing environment in the form of test code and flag configurations for subsequent execution of one or more algorithms to provide a results and/or generate a report in the reviewer's computing environment.

The application program for executing logic and/or algorithms may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described may either be part of the microinstruction code or part of the application program (or a combination thereof), which is executed via the operating system. In addition, various other peripheral devices may be connected to the computer platform such as an additional data storage device and a printing device. As a computer system, the system generally includes a processor unit. The processor unit operates to receive information, which generally includes test data (e.g., specific biomarkers assayed), and test result data (e.g., the AD biomarker expression for a sample). This information received can be stored at least temporarily in a database, and data analyzed in comparison to a library of AD biomarkers known to be at least partially indicative of the presence or absence of AD as described above.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In various embodiments, all or a portion of the input data and/or all or a portion of the output data (e.g., usually at least the library of AD biomarkers known to be at least somewhat indicative of the presence or absence of AD) are maintained on a server for access, preferably confidential access. The results may be accessed or sent to professionals as desired.

In various embodiments, a computer-readable storage medium (e.g. CD-ROM, memory key, flash memory card, diskette, etc.) is provided having stored thereon a program which, when executed in a computing environment, provides for implementation of the logic or algorithms to carry out all or a portion of the methods described herein, including detection of AD biomarkers.

Having now generally described the invention, the same may be more readily understood through the following reference to the following examples, which are provided by way of illustration and are not intended to limit the present invention unless specified.

### EXAMPLES

### Materials and Methods

Reagents for ELISAs were neutravidin-HRP, and QauntaBlu substrate kit (Pierce), PBS-Tween (Sigma), goat serum (Vector Laboratories), 96-well black Microfluor2-plates (Fisher Scientific), and curcumin (Cayman Chemicals). CSF samples were obtained from New York Brain Bank of Columbia University Medical Center. The samples were obtained from patients that had AD, severe AD or no AD. The definitive diagnosis was made by examination of tissue at autopsy.

The antibodies used in the examples described and the binding sites for the antibodies that were used to analyze various CSF samples are listed in Table A.

TABLE A

| Antibody | Epitope specificity | Application | Source |
|---|---|---|---|
| mAb HT7 | 159-163 | Capture antibody for total | Pierce (MN1000) |
| | (of Seq. ID No. 6) | tau, tau oligomer, glycated | |
| | | tau, (tau-Aβ1-42) assays. | |
| | | (FIGS.: 2, 7, 8, 10, 12-14.) | |
| Biotinylated mAb- | 159-163. | Reporter for modified | Pierce (MN1000B) |
| HT7 | (of Seq. ID No. 6) | ELISA (FIGS.: 7, 14) | |
| Biotinylated mAb | 194-198 | Reporter for standard | Pierce (MN1010 B) |
| BT2 | (of Seq. ID No. 6) | ELISA in total tau assay | |
| | | (FIG. 6) | |
| mAb T46 | 404-441 C-terminus | Capture antibody for tau- | Zymed/Invitrogen |
| | (of Seq. ID No. 6) | oligomer assay (FIGS. 9, 14) | ZY6400 |
| mAb TauC3 | Tau truncated at Asp | Capture antibody for | Chemicon (MAB5430) |
| | 421 | truncated tau oligomer | |
| | | assay.(FIG. 11) | |
| pAb | Tau phosphorylated at | Capture antibody for | Invitrogen (44-768G) |
| tau(pSpS¹⁹⁹/²⁰²) | Ser199 and Ser202 | tau(pSpS¹⁹⁹/²⁰²) (FIG. 3) | |
| pAb tau(pT²³¹) | Tau phosphorylated at | Capture antibody for | Invitrogen (44-746G) |
| | threonine 231 | tau(pT²³¹) assay (FIG. 5) | |
| pAb tau(pT²¹⁷) | Tau Phosphorylated | Capture antibody for | Invitrogen (44-744) |
| | at threonine 217 | tau(pT²¹⁷)assay (FIG. 4) | |
| Biotinylated pAb | Aβ1-42 | Reporter for (tau-Aβ1-42) | Invitrogen (44-3449) |
| (anti-Aβ1-42) | | assay (FIG. 12, 13) | |
| pAb AGE | Glycated tau | Capture antibody for | Abcam(ab23722) |
| | | glycated tau assay (FIG. 10) | |
| pAb A11 | Oligomer | Reporter for tau oligomer | Invitrogen (AHB0052) |
| | conformation | assay (FIG. 8, 9, 11, 14) | |
| | specific antibody | | |
| Biotinylated anti- | | Detection antibody when | Pierce(31820) |
| rabbit | | non-biotinylated rabbit | |
| | | antibody was used as | |
| | | reporter. (FIGS.: 8-11, 14) | |

### Example 1

Standard ELISA was performed to determine total tau levels in CSF. Total tau was determined by standard sandwich ELISA using mAb HT7 (tau epitope 159-163) for capture and biotinylated mAb BT2 (tau epitope 194-198) as reporter in a 96-well format as described above. The capture antibody was diluted to 5 μg/mL in sodium carbonate buffer pH 9.6 (coating buffer) and 100 μL was added to each well of a 96-well ELISA plate. The plate was sealed and incubated for 1 h at 37° C. The plate was washed three times with 300 μL of PBS-Tween (PBST) and blocked with 300 μL of 1% goat serum in PBST for 1 h at 37° C. The plate was washed again as mentioned previously.

CSF samples were diluted 10-fold in PBST and 100 μL of each sample was added to triplicate wells. Antibody blank wells received 100 μL of PBST. The plate was incubated for 1 h at 37° C., washed, and 50 μL of biotinylated BT7 (diluted 10,000-fold in PBST) was added to all wells. Plates were incubated at 37° C. for 1 hour, washed, and 50 μL of Neutravidin-HRP (diluted 3000-fold), was added to all wells, and plates were incubated for 30 min. at 37° C.

QuantaBlu™ fluorogenic substrate for HRP was prepared by mixing nine parts of QuantaBlu reagent with one part of H₂O₂ concentrate. The solution was allowed to stand at room temperature for equilibration. After washing the plate, 100 μL of substrate was added to each well and incubated at 37° C. for 30 min. The reaction was stopped by adding 100 μL of QuantaBlu stop solution and fluorescence was read in a Fluoroskan II fluorometer using 325 nm and 420 nm for excitation and emission wavelengths, respectively.

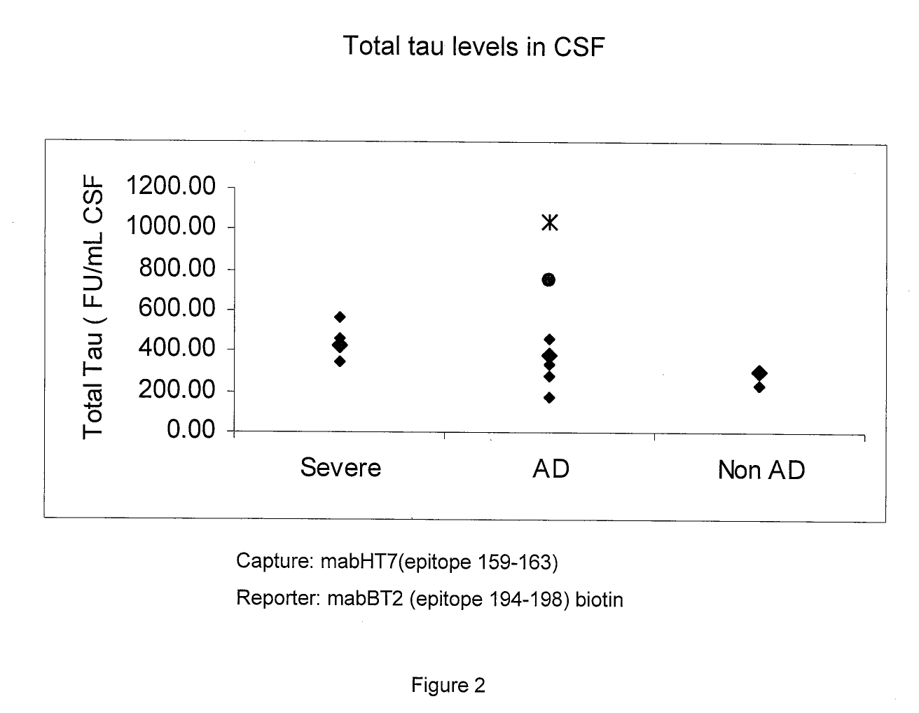

Data Analysis: (FU/mL CSF)=(Average fluorescence of triplicate wells for each sample−average fluorescence of the antibody blank wells)*CSF dilution factor. The results obtained are shown in FIG. 2 (4 severe AD, 7 AD and 3 non-AD CSF samples were tested). Here the total tau biomarker can discriminate non-AD from severe AD. This is a known assay platform and the other assay platforms with different and new biomarkers described and shown in FIGS. 4-5 and 7-14 can be validated using the data analysis method for the known biomarker total tau.

### Example 2

Standard ELISA to determine phosphorylated tau (pS199/202) levels in CSF was performed. Tau phosphorylated at S199/202 was determined by standard sandwich ELISA described for FIG. 2 (Example 1), except that rabbit pAb against tau pS199/202 (diluted 160-fold in coating buffer) was used for capture and biotinylated mAb HT7 (diluted 10,000-fold) was used as reporter. The results are shown in FIG. 3 (4 severe AD, 7 AD and 3 non-AD CSF samples were tested). Here tau phosphorylated at S199/202 biomarker can discriminate non-AD from severe AD. This is a known assay platform and the other assay platforms with different and new biomarkers described and shown in FIGS. 4-5 and 7-14 can be validated using the data analysis method for this known phosphorylated tau biomarker.

### Example 3

Standard ELISA to determine phosphorylated tau (pT217) levels in CSF. Tau phosphorylated at T217 was determined by standard sandwich ELISA described in FIG. 3 (Example 2), except that rabbit pAb against tau pT217 (diluted 300-fold in coating buffer) was used for capture. The results are shown in FIG. 4 (−4 severe AD, 7 AD and 3 non-AD CSF samples were tested). Here the novel observation is shown that tau phosphorylated at amino acid position 217 can be used as a biomarker to discriminate non-AD from severe AD.

### Example 4

Standard ELISA to determine phosphorylated tau (pT231) levels in CSF. Tau phosphorylated at T231 was determined by standard sandwich ELISA described for FIGS. 3 and 4, except that rabbit pAb against tau pT231 (diluted 160-fold in coating buffer) was used for capture. The results are shown in FIG. 5 (4 severe AD, 7 AD and 3 non-AD CSF samples were tested). Here tau was phosphorylated at amino acid position 231. This biomarker assay can discriminate non-AD from severe AD.

### Example 5

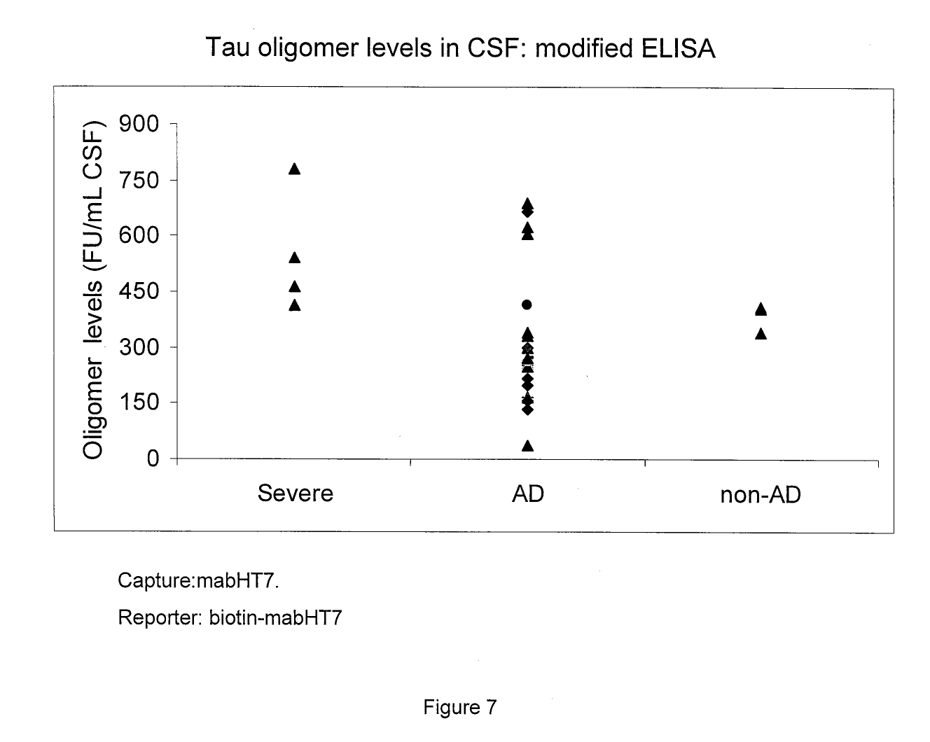

Modified ELISA to determine tau-oligomer levels in CSF: Tau oligomer level was determined using modified sandwich ELISA where mAb HT7 against tau was used for capture and biotinylated mAb HT7 as the reporter. The epitope for mAb HT7 is present only once in each tau protein. Here the capture antibody and the reporter antibody bind to the same epitope, and, thus, only detect tau oligomers which have multiple binding sites for mAb HT7. Detection was done using HRP-neutravidin and QuantaBlu fluorogenic substrate as described for FIG. 2 (Example 1). Here the assay interrogates soluble tau-oligomer using one antibody type. The results are shown in FIG. 7 (4 severe AD, 23AD and 3 non-AD CSF samples were tested). In severe AD, this biomarker may be increased when compared to AD and non-AD patients.

### Example 6

Tau oligomer levels based on oligomer conformation was determined using a standard sandwich ELISA where mAb HT7 against tau was used for capture, as mentioned in FIG. 2 (Example 1). After incubation with CSF samples (see FIG. 2 legend) the plates were washed and 100 uL rabbit pAb A11, specific for protein oligomer conformation, was diluted 1000-fold and added to each well. The plate was incubated for one hour at 37° C. and washed. Biotinylated goat anti-rabbit antibody was diluted 10,000-fold in PBST and 50 μL was added to each well. Plates were incubated for 30 min. at 37° C. Detection was done using HRP-neutravidin and QuantaBlu fluorogenic substrate as described for FIG. 2 (Example 1). The results are shown in FIG. 8 (4 severe AD, 5 AD and 3 non-AD CSF samples were tested). Here the assay is specific for tau in association with oligomer conformation and can detect soluble tau oligomers. The combination of a monoclonal antibody against tau with an antibody specific for oligomer conformation is novel. In severe AD, this biomarker may be increased when compared to AD and Non-AD patients.

### Example 7

Tau oligomer levels in CSF were determined using antibodies specific for oligomer conformation and tau with intact c-terminus. Assay was done as described for FIG. 8 (Example 6), except that mAbT46, specific for the 37 carboxyl terminal amino acids of tau (404-441 in SEQ ID NO: 6) was diluted 5000-fold in coating buffer and used for capture. Rabbit pAb A11, specific for oligomer conformation, was use for reporter. The results are shown in FIG. 9 (4 severe AD, 4 AD and 3 non-AD CSF samples were tested). Here the assay is specific for tau (with an intact c-terminus) in association with oligomer conformation and can detect soluble tau oligomers. The combination of a monoclonal antibody against tau (with an intact c-terminus) with an antibody specific for oligomer conformation is novel. This biomarker assay can discriminate non-AD from severe AD.

### Example 8

Glycated tau levels were determined by standard sandwich ELISA using mAb HT7 for capturing total tau and a rabbit pAb against AGE for reporter. The reporter antibody was diluted 3000-fold in PBST before use. Detection was done using biotin-goat anti-rabbit antibody, HRP-neutravidin and QuantaBlu fluorogenic substrate as described for FIG. 8 (Example 6). This assay platform is new and uses a monoclonal antibody specific for tau and an antibody that detects glycated proteins and can be used to detect glycated tau or tau in association with other glycated proteins. The results are shown in FIG. 10 (4 severe AD, 4 AD and 3 non-AD CSF samples were tested).

### Example 9

Truncated tau oligomer levels were determined by standard sandwich ELISA as described in FIG. 8 (Example 6), except mAb Tau C3, which detects truncated tau at Asp421, was used as the capture antibody. Here the assay determines levels of c-terminal truncated tau and utilizes a conformation specific antibody that detects soluble tau oligomer. The combination of a monoclonal antibody for truncated tau with an antibody specific for oligomer conformation is new. The results are shown in FIG. 11 (4 severe AD, 7 AD and 2 non-AD CSF samples were tested).

### Example 10

Tau-Aβ1-42 complex levels was determined by standard sandwich ELISA using mAb HT7 for capture of tau and biotinylated rabbit pAb against Aβ1-42 for reporter. Reporter antibody was diluted 5000-fold before use. Detection was done using HRP-neutravidin and QuantaBlu fluorogenic substrate as described for FIG. 7 (Example 5). The combination of a monoclonal antibody for soluble tau with an antibody specific for Aβ1-42 complex is new and can be used to detect complexes containing tau and Aβ1-42. The results are shown in FIG. 12 (4 severe AD, 7 AD and 1 non-AD CSF samples were tested). This biomarker assay can discriminate non-AD from severe AD.

### Example 11

### Curcumin Effect on Tau-Oligomer Levels in CSF

Sample preparation: A 5 mM stock solution of curcumin was prepared in 100% ethanol. The stock solution was diluted to 20 μM in buffer (50 mM Tris-HCL pH 7.4). Aliquots (50 μL) of CSF from three different severe AD patients were incubated for one hour at 37° C., with 5 uL of 20 uM curcumin, protease inhibitors, and 50 mM Tris-HCL pH 7.4 in a total volume of 100 uL. Controls contained CSF, protease inhibitor and 50 mM Tris-HCL pH 7.4 without curcumin. The incubated samples were diluted to 500 μL in buffer and analyzed for tau-oligomers by modified ELISA as described in FIG. 6. The results in FIG. 13 show curcumin's disruption of soluble tau oligomer. This assay can be used to screen compounds for disruption or modulation of soluble tau oligomer.

### Example 12

Curcumin effect on tau-oligomer levels in CSF: two assay formats are described. Sample preparation: Same as in Example 11, but the same CSF sample was used for both assays. The assay was performed as described in FIG. 7 (Example 5) for the modified ELISA. The standard ELISA was performed as described in FIG. 8 (Example 6).

Data analysis for Example 11 (FIG. 13) and Example 12 (FIG. 14) was measured as a percent decrease in tau-oligomers, upon incubation with curcumin. The calculation was as follows:

FU=(Average fluorescence of triplicate wells for each sample-average fluorescence of the antibody blank wells).
Percent decrease: 100*(FU of CSF incubated without curcumin−FU of CSF incubated with curcumin)/FU of CSF incubated without curcumin.
Here the results for standard ELISA gave similar results to the modified ELISA indicating that both assays can be used to validate curcumin's effect on soluble tau and to screen other compounds.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A method of determining the presence or absence of Alzheimer's disease (AD), the method comprising detecting in a sample a level of an AD biomarker comprising at least one phosphorylated tau pT217, soluble tau oligomer, tau-Aβ1-42 complex or a fragment thereof or a combination thereof; and comparing the level from the sample to a reference level of phosphorylated tau pT217, tau oligomer, and/or tau-Aβ1-42 to determine the presence or absence of AD.

2. A method of determining the presence or absence of AD according to claim 1, wherein the reference level is indicative of non-AD, mild AD, moderate AD, or severe AD.

3. A method of determining the presence or absence of AD according to claim 1, wherein the level of AD biomarker detected is above, equal or below the reference level, which indicates non-AD, AD, or severe AD.

4. A method of determining the presence or absence of AD according to claim 1, wherein the method utilizes one or more monoclonal and/or polyclonal antibodies or fragments thereof having one or more detectable moieties.

5. A method of determining the presence or absence of AD according to claim 4, wherein the detectable moiety comprises at least one radioactive, fluorescent, or enzymatically detectable label.

6. A method of determining the presence or absence of AD according to claim 1, wherein the level of AD biomarker is detected by: (1) immobilizing the sample to a support and contacting the sample with one or more labeled antibodies or fragment thereof that specifically bind to an epitope of phosphorylated tau pT217, tau oligomer, tau-Aβ1-42 complex, or a fragment thereof or a combination thereof; (i) measuring a signal attributable to bound labeled antibody or fragment thereof; and (ii) correlating the measured signal to the presence or amount of phosphorylated tau pT217, soluble tau oligomer, and/or tau-Aβ1-42 complex; or (2) immobilizing the sample to a support and contacting the sample with one or more antibodies or fragment thereof that specifically bind to an epitope of phosphorylated tau pT217, soluble tau oligomer, tau-Aβ1-42 complex, or a fragment thereof or a combination thereof; (i) contacting the bound antibodies or fragment thereof with a labeled antibody or fragment thereof that specifically binds to the bound antibody or fragment thereof; (ii) measuring a signal attributable to bound labeled antibody or fragment thereof; and (iii) correlating the measured signal to the presence or amount of phosphorylated tau pT217, soluble tau oligomer, and/or tau-Aβ1-42 complex; or (3) immobilizing an antibody or fragment thereof to a support and contacting the sample with the immobilized antibody or fragment thereof so as to specifically bind an epitope of phosphorylated tau pT217, soluble tau oligomer, tau-Aβ1-42 complex, or a fragment thereof or a combination thereof; (i) contacting the bound sample with a labeled antibody or fragment thereof that specifically binds to a second epitope of phosphorylated tau pT217, soluble tau oligomer, and/or tau-Aβ1-42 complex; (ii) measuring a signal attributable to bound labeled antibody or fragment thereof; and (iii) correlating the measured signal to the presence or amount of phosphorylated tau pT217, tau oligomer and/or tau-Aβ1-42 complex.

7. A method of diagnosing or stratifying AD according to claim 1, wherein the sample comprises cerebral spinal fluid or plasma.

8. A method of diagnosing, stratifying, or monitoring the progression or regression of Alzheimer's disease (AD), the method comprising detecting in a sample a level of at least one AD biomarker, the AD biomarker comprising at least phosphorylated tau pT217, soluble tau oligomer, tau-Aβ1-42 complex, a fragment thereof or a combination thereof and comparing the level from the sample to a reference level of phosphorylated tau pT217, tau oligomer, and/or tau-Aβ1-42 complex to diagnose or stratify or monitor the progression or regression of AD.

9. A method according to claim 8, wherein the reference level is indicative of non-AD, mild AD, moderate AD, or severe AD.

10. A method according to claim 8, wherein the AD biomarker level detected is above, equal or below the reference level, which indicates non-AD, AD, or severe AD.

11. A method of detecting or quantifying tau oligomer or tau-Aβ1-42 complex for use as a biomarker in Alzheimer's disease (AD), the method comprising contacting a sample with an antibody or fragment thereof and a labeled antibody or fragment thereof, the antibody or fragment thereof being capable of binding specifically to an epitope of tau or a fragment thereof and the labeled antibody or fragment thereof being capable of binding specifically to soluble tau oligomer, tau-Aβ1-42 complex or a fragment thereof or combination thereof; measuring a signal attributable to labeled antibody or fragment thereof bound to soluble tau oligomer or tau-Aβ1-42 complex; and correlating the measured signal to the presence or amount of soluble tau oligomer or tau-Aβ1-42 complex to use as a biomarker in AD.

12. A method of detecting or quantifying tau oligomer or tau-Aβ1-42 complex according to claim 11, wherein the biomarker is indicative of non-AD, AD, or severe AD.

13. A method of detecting or quantifying tau oligomer or tau-Aβ1-42 complex according to claim 11, wherein the antibody is a polyclonal antibody and the labeled antibody is a monoclonal antibody.

14. A method of detecting or quantifying tau oligomer or tau-Aβ1-42 complex according to claim 11, wherein a first antibody binds to a conformational epitope of soluble tau oligomer.

15. A method of detecting or quantifying tau oligomer or tau-Aβ1-42 complex according to claim 11, wherein the tau is phosphorylated, glycated tau, or truncated tau.

16. A method of detecting or quantifying tau oligomer or tau-Aβ1-42 complex according to claim 11, wherein the antibody or fragment thereof is immobilized to a solid support.

17. A method of detecting or quantifying soluble tau oligomer for use as a biomarker in Alzheimer's disease (AD), the method comprising contacting a sample with an antibody or fragment thereof and a labeled antibody or fragment thereof, the antibody or fragment thereof and the labeled antibody or fragment thereof being capable of binding specifically to the same epitope of soluble tau oligomer or a fragment thereof at different binding sites; measuring a signal attributable to bound labeled antibody or fragment thereof; and correlating the measured signal to the presence or amount of soluble tau oligomer to use as a biomarker in AD.

18. A method of detecting or quantifying tau oligomer according to claim 17, wherein the antibody or fragment thereof is immobilized to a solid support.

19. A method of detecting or quantifying tau oligomer according to claim 17, wherein the antibody or fragment thereof binds to one or more conformational epitopes of soluble tau oligomer and the labeled antibody or fragment thereof binds to the antibody or a second epitope of soluble tau oligomer.

20. A diagnostic kit or assay useful for detecting tau oligomer, tau-Aβ1-42 complex, and/or tau-Aβ1-40 complex in Alzheimer's disease (AD), the kit or assay comprising: an antibody being capable of binding specifically to an epitope of soluble tau and a labeled antibody being capable of binding specifically to a conformational epitope of soluble tau oligomer or an epitope of tau-Aβ1-42 or an epitope of tau-Aβ 1′-40 complex.

21. A diagnostic kit or assay according to claim 20, wherein the antibody is immobilized to a solid support.

22. A diagnostic kit or assay useful for detecting soluble tau oligomer in Alzheimer's disease (AD), the kit or assay comprising: a capture antibody and a labeled antibody, the capture antibody and the labeled antibody being the same antibody type and being capable of binding specifically to the same epitope of soluble tau oligomer at different sites.

23. A monoclonal antibody that binds specifically to phosphorylated tau pT217.

24. A method of screening an agent for modulation or disruption of soluble tau oligomer, the method comprising: a) contacting a sample containing soluble tau oligomer or a fragment thereof with an agent suspected of being capable of modulating tau oligomer formation or disrupting tau oligomers; and b) detecting the amount of soluble tau oligomer or fragment thereof in the sample, wherein a decrease in soluble tau oligomer indicates that the agent modulates tau oligomer formation or disrupts tau oligomer.

25. A method of screening an agent for modulation or disruption of tau oligomer according to claim 24, wherein the method further comprises c) comparing the sample to a sample that does not contain the agent or less agent than step a) to determine if the agent modulates or inhibits tau oligomer.

26. A method of screening an agent for modulation or disruption of tau oligomer according to claim 24, wherein the agent comprises curcumin, demethoxycurcumin, bis-demethoxycurcumin, and/or morin.

27. A method of screening an agent for modulation or disruption of tau oligomer according to claim 24, wherein the agent disrupts tau-tau oligomerizations by inhibiting tau-tau binding.

28. A method of monitoring a therapeutic effect of an agent that modulates or disrupts an AD (Alzheimer's disease) biomarker comprising soluble tau oligomer, tau-Aβ1-42 complex, tau-Aβ1-40 complex, and/or fragment thereof, the method comprising: a) contacting a sample containing the AD biomarker with an agent suspected of being capable of modulating or disrupting the AD biomarker; and b) detecting the amount of the AD biomarker in the sample, wherein an increase or decrease in the AD biomarker indicates that the agent modulates soluble tau oligomer, tau-Aβ1-42 complex, tau-Aβ1-40 complex, and/or fragment thereof.

29. A method of monitoring progression of AD (Alzheimer's disease) in a patient, the method comprising obtaining a sample of cerebral spinal fluid from the patient; detecting in the sample a level of an extracellular AD biomarker comprising soluble tau oligomer, tau-Aβ1-42 complex, tau-Aβ1-40 complex, and/or fragment thereof, and determining if there is an increase or decrease in the level of AD biomarker thereby monitoring the progression of AD.

30. A system, comprising: a computing environment; an input device, connected to the computing environment, to receive data from a user, wherein the data received includes a level of an Alzheimer's disease (AD) biomarker comprising at least one phosphorylated tau pT217, soluble tau oligomer, tau-Aβ1-42 complex or a fragment thereof or a combination thereof from a cerebrospinal fluid sample; an output device, connected to the computing environment, to provide information to the user; and a computer readable storage medium having stored thereon at least one algorithm to provide for comparing the AD biomarker from the cerebrospinal fluid to an AD biomarker known to be indicative of the presence or absence of AD.

31. A method according to claim 30, wherein the computing environment comprises a computer local to the user and a remote computer at a site remote to the user, wherein the local computer and the remote computer are connected through a network, and wherein the computer readable storage medium is provided on the remote computer.
